(19) United States
(12) Patent Application Publication
(10) Pub. No.: US 2024/0058295 A1
(43) Pub. Date: Feb. 22, 2024

(54) TREATING IRON DEFICIENCY WITH FERRIC CARBOXYMALTOSE

(71) Applicant: PHARMACOSMOS HOLDING A/S, HOLBAEK (DK)

(72) Inventors: Tobias Sidelmann CHRISTENSEN, Roskilde (DK); Philip SCHAFFALITZKY DE MUCKADELL, Virum (DK); Lars Lykke THOMSEN, Holte (DK); Claes Christian STROM, Rungsted (DK)

(21) Appl. No.: 18/370,926

(22) Filed: Sep. 21, 2023

Related U.S. Application Data
(62) Division of application No. 17/187,992, filed on Mar. 1, 2021, now Pat. No. 11,806,329.
Application No. 17/187,992 is a division of application No. 16/822,911, filed on Mar. 18, 2020, now Pat. No. 11,020,369.
Application No. 16/822,911 is a division of application No. PCT/EP2019/079528, filed on Oct. 29, 2019.

(30) Foreign Application Priority Data
Oct. 29, 2018 (EP) 18203223.5
Oct. 31, 2018 (EP) 18203818.2

(51) Int. Cl. A61K 31/295; A61P 7/06

(52) U.S. Cl. CPC A61K 31/295; A61P 7/06

(57) ABSTRACT

The present invention relates to the field of treating iron deficiency with IV iron carbohydrate complexes such ferric carboxymaltose, monitoring or identifying subjects to determine their eligibility for being administered said IV iron carbohydrate complexes, and combining said IV iron carbohydrate complexes with additional drugs in order to mitigate or reduce side effects induced by said IV iron carbohydrate complexes.

This application is a divisional application of U.S. application Ser. No. 17/187,992 filed Mar. 1, 2021, which is a divisional application of U.S. application Ser. No. 16/822,911, filed Mar. 18, 2020, now U.S. Pat. No. 11,020,369, which is a divisional of Patent Cooperation Treaty (PCT) Appl. No. PCT/EP2019/079528 filed Oct. 29, 2019, which claims priority to European Patent Application Nos. EP 18203223.5, filed Oct. 29, 2018 and EP 18203818.2, filed Oct. 31, 2018, each of which is hereby incorporated by reference herein in its entirety.

### FIELD OF THE INVENTION

The present invention relates to the field of treating iron deficiency with IV iron carbohydrate complexes, monitoring or identifying subjects to determine their eligibility for being administered said IV iron carbohydrate complexes, and combining said IV iron carbohydrate complexes with additional drugs in order to mitigate or reduce side effects induced by the IV iron carbohydrate complexes.

### BACKGROUND

Iron deficiency (ID) impairs the body's ability to produce hemoglobin, the key oxygen transporter, and impairs the function of key energy (ATP) producing enzymes. Symptoms consequently include fatigue and other signs of energy deprivation such as rapid heartbeat, shortness of breath, and chest pain.

ID has serious consequences. In chronic heart failure (CHF) patients, the risk of death or hospitalization is increased in patients with ID relative to patients with normal iron status. Quality of life (QoL) is severely affected and improves rapidly upon restoration of iron stores. Patients with ID or IDA undergoing surgery have poor outcomes—in part due to greater risk of blood transfusions. Maternal iron deficiency is associated with increased risk of pre-term birth and impaired fetal brain development.

Iron deficiency anemia (IDA) develops when iron stores are depleted. It is widespread. About 1 billion people worldwide suffer from IDA according to the WHO. 4.5 million patients are diagnosed with IDA Daily oral iron is the first line therapy for most IDA patients but often fails due to lack of compliance, lack of efficacy and side effects.

High dose intravenous (IV) iron is an attractive treatment option. Patients typically require 1-3 grams of iron per year and high dose IV iron effectively and rapidly improves symptoms and increases hemoglobin levels. High dose IV iron allows treatment in one or few visits and IV iron is the only option for patients failing oral iron.

Ferric carboxymaltose (FCM) belongs to a new generation of high dose IV iron products. While older low dose products (ferric gluconate and iron sucrose) required 5-20 visits, these new generation products allow for iron correction in one or two visits by fast infusion of the product.

Moderate and transient decreases in serum phosphate (S-phosphate) have been observed for all iron complexes upon IV administration in humans to treat iron deficiency or iron deficiency anemia. This general phenomenon is believed by some to be associated with the consumption of phosphate in erythropoiesis, a primary intended effect of parenteral iron therapy. See, for instance, Van Wyck et al., 2009. Others have favored a theory of phosphate wasting which postulates that the renal phosphate loss could be the consequence of proximal tubular dysfunction due to a direct toxic effect of IV iron on proximal renal tubular cells. Prats et al., 2013.

Ferric carboxymaltose (FCM) is a very commonly used iron carbohydrate complex to treat patients with ID or IDA who are not on dialysis. It is commercially available in the United States under the tradename Injectafer® and in the European Union and many other countries under the tradename Ferinject®. A typical treatment regimen of FCM consists either of two doses of 750 mg of elemental iron given as intravenous infusion one week apart (this is the approved use according to the US label) or as an infusion of 1000 mg of elemental iron followed by an additional dose of 500-1000 mg one week after (this is the approved use according to its EU label).

FCM has been shown to lead to a larger and longer reduction in serum phosphate as compared to iron dextran (Wolf et al., 2013), iron sucrose (WO2013/134273 A1) and iron isomaltoside 1000 (Bager et al., 2016, Schaefer et al., 2016, Zoller et al., 2017) and as a result a higher prevalence of hypophosphatemia, i.e., the condition characterized by too low serum phosphate.

Nonetheless, although there are individual case reports on hypophosphatemia resulting from the treatment with FCM (Anand G, Schmid C, BMJ Case rep 2017) and subsequent bone complications such as osteomalacia occurring months thereafter (see, for instance, Schaefer et al. 2017; Klein et al. 2018), such findings have been actively disputed by the majority of publications or characterized as so rare to be a curiosity. A large number of scientific publications on the use of FCM describe how the associated hypophosphatemia is considered to be transient, asymptomatic and/or clinically irrelevant. See, for instance, Aksan et al., 2007; Bregman et al., 2014; Charytan et al., 2013; Evstatiev, 2011; Hussain et al., 2013; Ikuta et al., 2018; Prats et al., 2013; Qunibi et al., 2011; Sari et al., 2017; Seid et al., 2008; Stein et al., 2018; Van Wyck et al., 2009. This perspective has so far been supported by the absence of studies demonstrating any short-term clinical impacts of the lowered phosphate levels despite the changes in biochemical parameters.

US and EU regulators have so far also taken the position that FCM-associated hypophosphatemia is transient, asymptomatic and clinically irrelevant. Although the U.S. FDA in a 2007 non-approval letter listed clinically important hypophosphatemia as one of three potential safety risks that would need to be resolved through additional clinical data in order to verify the safety of the product, the subsequent submission of data by the sponsor of Injectafer® led the agency to conclude in 2013 that all the clinical (efficacy and safety) issues brought forth in the non-approval letter including the issue of clinically important hypophosphatemia had been satisfactorily resolved (U.S. Federal Drug Administration Center for drug evaluation and research, application number: 2035650rig1s000, summary review 2013). The view that hypophosphatemia associated with FCM is mild and transient is also reflected in the currently approved labels, which list hypophosphatemia as a side effect, but do not provide particular warnings related to hypophosphatemia, neither in terms of short- or long-term consequences. On the contrary, aside from listing it as a side effect the only mention of hypophosphatemia in the EU Summary of Product Characteristics (SmPC) which forms part of the regulatory approval of FCM in Europe is the following statement: “In clinical trials, the minimum serum phosphorous values were obtained after approximately 2 weeks, and 4 to 12 weeks following Ferinject treatment the values had returned to those within the range of baseline”.

Fibroblast growth factor 23 (FGF23) is an osteocyte-derived hormone that regulates phosphate and vitamin D homeostasis. It undergoes proteolytic cleavage and as a result a mix of uncleaved, i.e. intact FGF23 (iFGF23), and its cleavage fragments are found in vivo. Because reduced serum phosphate in response to intravenous iron was suggested to be mediated by an acute increase in FGF23, Wolf et al. examined the effects of iron deficiency and its rapid correction on C-terminal and intact FGF23 levels in women with iron deficiency anemia secondary to heavy uterine bleeding. Their findings suggested that iron deficiency increases C-terminal FGF23 (cFGF23) levels, and that FCM temporarily increased iFGF23 levels and reduced serum phosphate. Wolf et al., 2013; WO2013/134273 A1.

We have surprisingly found that contrary to the general understanding in the art, treatment with FCM according to current practice leads to direct clinical consequences such as reduced muscle function and increased bone turnover. Furthermore, we have found that the current practice related to repeated dosing of FCM one week apart leads to an auto-synergistic impact on iFGF23, with the second dose leading to a 2-3 fold higher increase than the first dose.

Based on this understanding, there is clearly a need for improved methods of using FCM in the treatment of the underlying ID or IDA, which methods substantially decrease the risk of iFGF23-induced consequences such as reduced muscle function and increased bone turnover. These and other iFGF23-induced metabolic, nutritional and musculoskeletal consequences of FCM treatment are hereinafter referred to as the iFGF23-mediated or iFGF23-induced side effects.

### SUMMARY

In one aspect of this invention, the treatment with ferric carboxymaltose can be completed without loss of efficacy, but with a reduced risk of iFGF23-mediated side effects by adjusting the timing and/or the amount of FCM administered in order to avoid auto-synergistic effects.

In a second aspect of this invention, patients are selected for treatment with ferric carboxymaltose not only based on the criteria commonly used to define eligibility for IV iron, i.e. diagnosis of ID or IDA and a potential lack of the ability to tolerate or absorb oral iron, but also based on being less likely to suffer from iFGF23-mediated side effects.

In a third aspect of this invention, a subject who has been administered a first dose of ferric carboxymaltose is monitored to determine if or when the subject is eligible for being administered a second dose of ferric carboxymaltose.

In a fourth aspect of this invention, a subject having a reduced risk for FGF23-mediated side effects is identified.

In a fifth aspect of this invention, ferric carboxymaltose is combined with supporting drugs to mitigate or reduce the impact of iFGF23-mediated side effects.

In line with these aspects, the present invention in particular relates to therapeutic methods of treating iron deficiency which comprise administering ferric carboxymaltose according to defined regimens and/or to selected subgroups of subjects; diagnostic methods for monitoring subjects who have been administered a first dose of FCM to adjust the timing and/or the amount of further FCM administration, or for identifying subjects suitable for the therapeutic methods of the invention; and combinations of FCM with other drugs that mitigate or reduce the impact of iFGF23-mediated side effects.

In a first embodiment of said first aspect, the present invention relates to a method of treating iron deficiency, which comprises administering a first dose and a second dose of ferric carboxymaltose, wherein the time between the first and the second dose is at least 10 days.

In a second embodiment of said first aspect, the present invention relates to a method of treating iron deficiency, which comprises administering a first dose and a second dose of ferric carboxymaltose, wherein the first and the second dose each do not exceed 500 mg of elemental iron.

In a third embodiment of said first aspect, the present invention relates to a method of treating iron deficiency, which comprises administering one or more doses of ferric carboxymaltose, wherein the total amount of elemental iron administered within a period of 12 months does not exceed 5000 mg.

In a first embodiment of said second aspect, the present invention relates to a method of treating iron deficiency which comprises administering ferric carboxymaltose, wherein the subject having a reduced risk for FGF23-mediated side effects has blood parameters as disclosed herein.

In a second embodiment of said second aspect, the present invention relates to a method of treating iron deficiency which comprises administering ferric carboxymaltose, wherein the subject having a reduced risk for FGF23-mediated side effects is characterized by the absence of exclusion criteria as disclosed herein.

In a third embodiment of said second aspect, the present invention relates to a method of treating iron deficiency which comprises administering ferric carboxymaltose, wherein the subject having a reduced risk for FGF23-mediated side effects is characterized by respiratory capacity as disclosed herein.

In a first embodiment of said third aspect, the present invention relates to a method of monitoring a subject who has been administered a first dose of ferric carboxymaltose, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject is eligible for being administered a second dose of ferric carboxymaltose if the at least one blood or urine parameter is as disclosed herein.

In a second embodiment of said third aspect, the present invention relates to a method of monitoring a subject who has been administered a first dose of ferric carboxymaltose, comprising determining in a biological sample obtained from the subject at least one blood parameter selected from the group consisting of (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject is eligible for being administered a second dose of ferric carboxymaltose if the at least one blood parameter is as disclosed herein.

In a third embodiment of said third aspect, the present invention relates to a method of monitoring a subject who has been administered a first dose of ferric carboxymaltose, comprising determining the respiratory capacity of the subject, wherein the subject is eligible for being administered a second dose of ferric carboxymaltose if the respiratory capacity is as disclosed herein.

In a first embodiment of said fourth aspect, the present invention relates to a method of identifying a subject having a reduced risk for FGF23-mediated side effects, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject has a reduced risk for FGF23-mediated side effects if the at least one blood or urine parameter is as disclosed herein.

In a second embodiment of said fourth aspect, the present invention relates to a method of identifying a subject having a reduced risk for FGF23-mediated side effects, comprising determining in a biological sample obtained from the subject at least one blood parameter selected from the group consisting (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject has a reduced risk for FGF23-mediated side effects if the at least one blood parameter is as disclosed herein.

In a third embodiment of said fourth aspect, the present invention relates to a method of identifying a subject having a reduced risk for FGF23-mediated side effects, comprising determining whether the subject is characterized by one or more and in particular all of the following exclusion criteria:
(1) having undergone bariatric surgery;
(2) obesity;
(3) cardiac conditions with increased risks of arrhythmias;
(4) primary or secondary hyperparathyroidism;
(5) pulmonary disorders such as asthma or chronic obstructive pulmonary disease (COPD)
(6) genetic diseases leading to hypophosphatemia such as X-linked hypophosphatemia, autosomal dominant hypophosphatemic rickets, autosomal recessive hypophosphatemic rickets;
(7) secondary hypophosphatemia or tumor induced hypophosphatemia;
(8) disorders of the bone, such as for example osteoporosis or osteomalacia; and
(9) being scheduled for surgery within 1 day to two months of the iron administration, wherein the subject has a reduced risk for FGF23-mediated side effects if the subject is characterized by the absence of one or more and in particular all of the exclusion criteria.

In a fourth embodiment of said fourth aspect, the present invention relates to a method of identifying a subject having a reduced risk for FGF23-mediated side effects, comprising determining the respiratory capacity of the subject, wherein the subject has a reduced risk for FGF23-mediated side effects if the respiratory capacity is as disclosed herein.

In a first embodiment of said fifth aspect, the present invention relates to a combination of ferric carboxymaltose with one or more additional drugs selected from the group consisting of:
(1) vitamin Ds, such as calcitriol;
(2) phosphates, such as glucose-1-phosphate, calcium phosphate or sodium phosphate; and
(3) anti-FGF23 antagonistic antibodies, such as burosumab.

### BRIEF DESCRIPTION OF THE DRAWINGS

FIG. 1 is a table summarizing the analysis of the occurrence of hypophosphatemia (primary endpoint) for study IDA-04.

FIG. 2 is a table summarizing the analysis of the occurrence of hypophosphatemia (primary endpoint) for study IDA-05.

FIG. 3 shows the incidence of phosphate levels≤2.0 mg/dL by visit (IDA-04 and IDA-05 combined; FCM: left-hand bars, IIM: right-hand bars).

FIG. 4 shows the incidence of phosphate levels≤1.0 mg/dL by visit (IDA-04 and IDA-05 combined; FCM: left-hand bars, IIM: right-hand bars).

FIG. 5 shows the mean absolute phosphate levels over time (IDA-04 and IDA-05 combined).

FIG. 6 shows the change in iFGF23 over time (IDA-04).

FIG. 7 shows the change in iFGF23 over time (IDA-05).

FIG. 8 shows the change in cFGF23 over time (IDA-04).

FIG. 9 shows the change in cFGF23 over time (IDA-05).

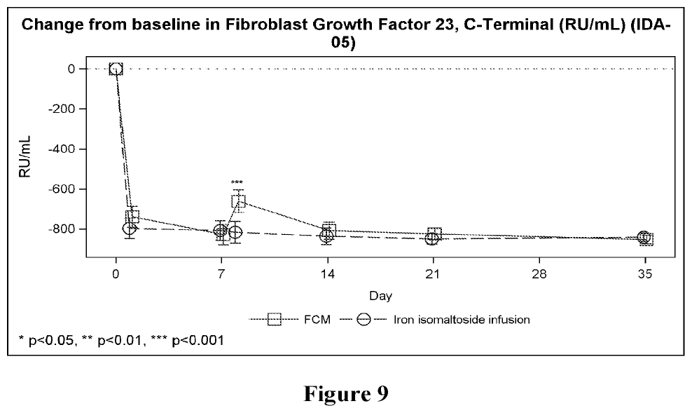

FIG. 10 shows the change of fractional urinary phosphate excretion over time (IDA-04 and IDA-05 combined).

FIG. 11 shows the mean change from baseline and absolute change of vitamin D levels over time (IDA-04 and IDA-05 combined).

FIG. 12 shows the change from baseline and absolute change of intact parathyroid hormone levels overtime (IDA-04 and IDA-05 combined).

FIG. 13 shows the change from baseline and absolute change of calcium ions over time (IDA-04 and IDA-05 combined).

FIG. 14 shows the mean change of several makers of muscle function and bone turnover over time (IDA-04 and IDA-05 combined).

FIG. 15 shows the mean change in respiratory muscle strength over time (IDA-04 and IDA-05 combined).

### DETAILED DESCRIPTION

Described herein are therapeutic methods of treating iron deficiency which comprise administering an iron carbohydrate complex, methods of monitoring a subject who has been administered a first dose of an iron carbohydrate complex, methods of identifying a subject suitable for the therapeutic methods of the invention, and combinations of an iron carbohydrate complex with additional drugs, wherein the iron carbohydrate complex induces a significant (e.g., statistically significant) increase of iFGF23 levels in subjects under treatment. The methods of the invention are thus applicable to complexes that share the mechanism of inducing significant increases in iFGF23 and which, as a result, can reduce serum phosphate levels and thus lead to hypophosphatemia. While this does not include the other IV iron drugs commonly used in Europe and the US, (such as iron sucrose (Venofer®), iron gluconate (Ferrlecit®), iron isomaltoside 1000 (Monofer®), or iron dextran (Cosmofer®/INFeD® and Dexferrum®), it does include certain complexes available in Asia-Pacific including iron saccharated oxide available in Japan as Fesin® (which despite similarity in name is distinct from iron sucrose in Venofer®), some species of iron polymaltose and above all ferric carboxymaltose (FCM). Results from clinical trials and case reports suggest that the highest risk for an iFGF23-mediated reduction of serum phosphate levels and the development of hypophosphatemia is associated with iron polymaltose, saccharated iron oxide, and above all ferric carboxymaltose. See, for instance, Wolf et al., 2013.

Accordingly, the preferred iron carbohydrate complex of this invention is ferric carboxymaltose (FCM). The term “ferric carboxymaltose” as used herein refers to colloidal complexes comprising iron, e.g., as iron oxide hydroxide, and carboxymaltose. Carboxymaltose is based on starch or starch derivatives that have been carboxylated, i.e., modified to include carboxy groups, for example through oxidation of the aldehyde end groups. A particular ferric carboxymaltose is obtainable by oxidizing maltodextrin, as described, for instance, in WO2007/081744 A1 as VIT-45 and WO2004/037865 A1. A preferred example of ferric carboxymaltose is commercially available in the United States under the tradename Injectafer® and in the European Union and many other countries under the tradename Ferinject®.

### Definitions

In order that the present description may be more readily understood, certain terms are first defined. Additional definitions are set forth throughout the detailed description.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

A “therapeutically effective amount” or “therapeutically effective dose” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Fibroblast growth factor 23 (FGF23)” is an osteocyte-derived hormone that regulates phosphate and vitamin D homeostasis. FGF23 undergoes proteolytic cleavage and as a result a mix of uncleaved, i.e. intact FGF23 (iFGF23), and its cleavage fragments are found in vivo. The intact form, iFGF23, is the active form in relation to phosphate metabolism where it controls the urinary excretion of phosphate, with increasing levels of iFGF23 leading to urinary wasting of phosphate. Two main types of antibody assays currently exist, one which captures only iFGF23 and another which binds to the C-terminal end of the hormone and therefore captures both iFGF23 and C-terminal fragments. The latter metric, cFGF23, is therefore a measure of the sum of intact FGF23 and C-terminal FGF23 fragments. Thus, two test related to FGF23 exist, iFGF23 and cFGF23, which have different interpretations.

“Hypophosphatemia” is a condition characterized by too low serum phosphate levels. The Common Terminology Criteria for Adverse Events (CTCAE) Version 4.0 provides four grades of hypophosphatemia.

| Grade 1 | Grade 2 | Grade 3 | Grade 4 | Grade 5 |
|---|---|---|---|---|
| <LLN-2.5 | <2.5-2.0 | <2.0-1.0 | <1.0 | Death |
| mg/dL | mg/dL | mg/dL | mg/dL | |
| <LLN-0.8 | <0.8-0.6 | <0.6-0.3 | <0.3 | |
| mmol/L | mmol/L | mmol/L | mmol/L | |

LLN is the lower limit of normal range used by a specific laboratory.

As used herein, unless specified otherwise, frequencies of hypophosphatemia refer to serum phosphate levels below 2 mg/dL.

The term “serum phosphate (S-phosphate)” as used herein refers to the level of inorganic phosphorus in serum blood as measured as an ammonium phosphomolybdate complex having the formula (NH₄)₃[PO₄](MoO₃)₁₂ formed by the reaction of inorganic phosphorous with ammonium molybdate in the presence of sulfuric acid. The complex is determined photometrically in the ultraviolet region (340 nm) of the spectrum using the Roche Modular and Cobas Analyzers.

The term “serum vitamin D” as used herein refers to the level of vitamin Ds, in particular 25-hydroxyvitamin D, 1,25-dihydroxyvitamin D, and 24,25-dihydroxyvitamin D, in blood serum as measured by Liquid Chromatography and Tandem Mass Spectrometry (LC-MS/MS).

The term “serum ionized calcium” as used herein refers to the level of calcium ions (Ca²⁺) in blood serum as measured using the IL GEM Premier 3500 PAK cartridge. The central component is the sensor card, which provides a low volume, gas tight chamber in which the blood sample is presented to the sensors. The pH and electrolyte sensors are all based on the principle of ion-selective electrodes; that is, an electrical potential can be established across a membrane which is selectively permeable to a specific ion. The pH and electrolyte sensors are polyvinyl chloride (PVC) based ion selective electrodes, consisting of an internal Ag/AgCl reference electrode and internal salt layer. The potentials are measured against the card reference electrode.

The term “serum parathyroid hormone (PTH)” as used herein refers to the level of serum parathyroid hormone in blood serum as measured using a two-site sandwich immunoassay using direct chemiluminometric technology, which used constant amounts of two anti-human PTH antibodies. PTH, produced by the parathyroid gland, is the major circulating factor regulating extracellular calcium concentration. Abnormally low ionized calcium concentrations trigger the secretion of PTH. The PTH molecules bind to type 1 PTH receptors in target tissues and initiate a sequence of reactions that results in an increase in extracellular calcium concentrations. PTH stimulates osteoclastic bone resorption resulting in the release of calcium from bone. PTH stimulates transcellular calcium reabsorption from the renal tubules and stimulates the kidney to produce 1,25-dihydroxyvitamin D, which acts on the intestines to increase calcium reabsorption. In most clinical conditions, rising levels of extracellular calcium will suppress PTH secretion through a negative feedback mechanism.

The term “fractionary urinary excretion of phosphate (FEPi)” (sometimes abbreviated FEPO4) is a measure of how much phosphate is not re-absorbed from the pre-urine in the kidneys, i.e., how much phosphate ends up in the urine as a proportion of how much phosphate the subject has in the blood. It is calculated like this: FEPi=[PO4 (Urine)*Creatinine (Serum)]/[PO4 (Serum)*Creatinine (Urine)]*100. An FEPi of 10-20% (0.1-0.2 fraction) is usually considered to be normal; an FEPi<10% (<0.1 fraction) is usually considered to be low; and an FEPi>20% (>0.2 fraction) is usually considered to be high. Phosphate in urine is measured using the same methodology as for serum.

The term “serum Bone Specific Alkaline Phosphatase” as used herein refers to the level of Bone specific Alkaline Phosphatase in blood serum as measured using the Beckman-Coulter Ostase assay which is a one-step immunoenzymatic chemiluminescence assay using a mouse monoclonal antibody specific to Bone Specifc Alkaline Phosphatase (BAP).

The term “serum Alkaline Phosphatase” as used herein refers to the level of Alkaline Phosphatase in blood serum as measured enzymatically using a Roche Modular Analyzer. In the presence of magnesium and zinc ions, p-nitrophenyl phosphate is cleaved by phosphatase into phosphate and p-nitrophenol. The p-nitrophenol released is proportional to the Alkaline Phosphatase activity and is measured photometrically.

The term “serum N-terminal Propeptide of Type I Collagen (PINP)” as used herein refers to the level of PINP in blood serum as measured using a sandwich principle, electrochemiluminescence immunoassay (ECLIA) on a Cobas e601 Analyzer. During the first incubation, PINP in the sample and a biotinylated monoclonal PINP-specific antibody are incubated together. During the second incubation, streptavidin-coated labelled microparticles and a monoclonal PINP-specific antibody labelled with ruthenium complex (Trs (2,2-bipyridyl)ruthenium (II)-complex (Ru(bpy)23+)) are added to form a sandwich complex, which binds to the solid phase via interaction of biotin and streptavidin. The reaction mixture is aspirated into the measuring cell where the microparticles are magnetically captured onto the surface of the electrode. A voltage is applied to the electrode, which induces chemiluminescent emission, which is measured by a photomultiplier. Results are determined via a calibration curve, which is instrument-specifically generated by a 2-point calibration and a master curve provided via the reagent barcode.

The term “serum Carboxy-terminal Collagen Crosslinks (CTx)” as used herein refers to the level of CTx in blood serum as measured using a sandwich principle, electrochemiluminescence immunoassay (ECLIA) on a Cobas e601 Analyzer. During the first incubation, 50 μL of sample and biotinylated monoclonal anti-beta-CrossLaps antibodies are incubated together. During the second incubation, streptavidin-coated labelled microparticles and a monoclonal P-beta-CrossLaps-specific antibody labelled with ruthenium complex are added to form a sandwich complex, which binds to the solid phase via interaction of biotin and streptavidin. The reaction mixture is aspirated into the measuring cell where the microparticles are magnetically captured onto the surface of the electrode. Unbound substances are then removed with ProCell. A voltage is applied to the electrode, which induces chemiluminescent emission, which is measured by a photomultiplier. Results are determined via a calibration curve, which is instrument-specifically generated by a 2-point calibration and a master curve provided via the reagent barcode.

The term “serum creatine kinase (CK)” as used herein refers to the CK level in blood serum as measured enzymatically using a Roche Modular and Cobas Analyzers. CK catalyzes the phosphorylation of ADP by creatine phosphate. ATP is performed which phosphorylates glucose and the resulting glucose-6-phosphate converts NADP+ to NADPH. The rate of NADPH formation is proportional to CK activity and is measured photometrically. CK is an enzyme that catalyzes the reversible transfer of phosphate from ATP to Creatine. This makes possible the storage of high-energy phosphate in a more stable form in ATP. CK is present in high concentration in skeletal muscle, cardiac muscle, thyroid, prostate and brain; it is present only in small amounts in liver, kidney, lung and other tissues. Hence, an increase in serum CK activity is ascribed primarily to damage to striated muscle (skeletal or cardiac) and in rare cases, to brain. Differentiation of these various diseases can frequently be made upon clinical grounds, but there are situations when this is not possible. Measurement of CK isoenzymes helps solve the problem.

The term “serum Ferritin” as used herein refers to the level of Ferritin in blood serum as measured using a two-site immunoenzymatic (“sandwich” assay). Ferritin is the major iron storage protein for the body. The concentration of ferritin is directly proportional to the total iron stores of the body, resulting in serum ferritin levels becoming a common diagnostic tool in the evaluation of iron status. Patients with iron deficiency anemia have serum ferritin levels approximately one tenth of normal subjects, while patients with iron overload (hemochromatosis, hemosiderosis) have serum ferritin level much higher than normal.

Ferritin levels also provide a sensitive means of detecting iron deficiency at an early stage. In both adults and children, chronic inflammation results in a disproportionate increase in ferritin levels in relation to iron reserves. Elevated ferritin levels also are observed in acute and chronic liver disease, chronic renal failure and in some types of neoplastic disease.

It is noted that while the above blood parameters are determined in serum, they can likewise be determined in plasma. Serum and plasma levels correlate and can be converted into each other.

The term “bariatric surgery” as used herein refers to a surgical procedure aiming at introducing weight loss by reducing the size of the stomach with a gastric band or removal of a portion of the stomach (sleeve gastrectomy or biliopancreatic diversion with duodenal switch) or by resecting and re-routing the small intestine to a small stomach pouch.

“Obesity” is a condition characterized by excessive body weight to the extent when the body mass index (BMI), a measurement obtained by dividing a person's weight by the square of the person's height, is over 30 kg/m².

“Cardiac conditions with increased risks of arrhythmias” are conditions characterized by cardiovascular conditions and risk factors that increase the chance of developing arrhythmias, or abnormal heart rhythms including but not limited to: Coronary artery disease, endocarditis, valvular heart disease, high blood pressure, diabetes and obesity.

“Primary or secondary hyperparathyroidism” is a condition characterized by excessive secretion of parathyroid hormone.

“Asthma” is a condition characterized by a chronic lung disease that inflames and narrows the airways and causes recurring periods of wheezing, chest tightness, shortness of breath, and coughing.

“Chronic obstructive pulmonary disease (COPD)” is a condition characterized by a chronic inflammatory lung disease that causes obstructed airflow from the lungs.

“X-linked hypophosphatemia” is a condition characterized by a hereditary renal phosphate-wasting disorder characterized by low levels of phosphate in blood, rickets and/or osteomalacia, and diminished growth.

“Autosomal dominant or recessive hypophosphatemic rickets” is a condition characterized by bones that become soft and bend easily, due to low levels of phosphate in the blood.

“Secondary hypophosphatemia” is a condition characterized by low levels of phosphate in blood due to decreased oral intake or decreased intestinal absorption.

“Tumor induced hypophosphatemia” is a condition characterized by low levels of phosphate in blood secondary to tumor, such as FGF23 producing tumors.

“Osteoporosis” is a condition characterized by a bone disease that occurs when the body loses too much bone, makes too little bone, or both. As a result, bones become weak.

“Osteomalacia” is a condition characterized by the softening of the bones caused by impaired bone metabolism primarily due to inadequate levels of available phosphate, calcium, and vitamin D, or because of resorption of calcium. The impairment of bone metabolism causes inadequate bone mineralization.

A subject “being scheduled for surgery within 1 day to two months of the iron administration” is a subject who will have surgery within 1 day to two months of the iron administration.

For the purpose of this text, when specifying a dose in mg or g of an iron carbohydrate complex, consistent with the practice in the literature, the value refers to the amount of elemental iron provided in mg.

### A. Therapeutic Methods

Described herein are therapeutic methods of treating iron deficiency which comprise administering ferric carboxymaltose according to defined administration regimens and/or to selected subgroups of subjects. Accordingly, the present invention also relates to ferric carboxymaltose for use in said methods, the use of ferric carboxymaltose for treating iron deficiency and or the use of ferric carboxymaltose in the manufacture of a medicament for treating iron deficiency.

I. Administration regimens

According to a first embodiment of this aspect of the invention, the method of treating iron deficiency comprises repeated administration of the iron carbohydrate complex, in particular FCM, which is characterized in that the time between the first and the second dose is at least 10 days. For instance, if the first dose is administered at day 0, the second dose is administered at day 10 or thereafter. Preferably, the time between the first and the second dose is at least 14 days, 18 days, 21 days, 28 days, 35 days, 42 days, 49 days, or 56 days. In some cases, it can even be expedient that the time between the first and the second dose is at least 3 months or 6 months.

The daily dose of elemental iron administered is a therapeutically effective amount that may be in the range of 500 mg to 1000 mg, e.g. at 500 mg, 750 mg, or 1000 mg elemental iron.

The dose is in particular a single daily dose. For instance, a typical single daily dose of ferric carboxymaltose is 500 mg, 750 mg or 1000 mg elemental iron. For repeated administration, a first dose of 750 mg elemental iron is followed by a second dose of 750 mg elemental iron, or a first dose of 1000 mg elemental iron is followed by a second dose of 500 mg to 1000 mg of elemental iron, e.g. 500 mg, 750 mg or 1000 mg of elemental iron. Further doses of FCM may follow.

According to a second embodiment of this aspect of the invention, the method of treating iron deficiency comprises repeated administration of the iron carbohydrate complex, in particular FCM, which is characterized in that each of the first and the second dose does not exceed, or is less than, 500 mg of elemental iron. Exemplary amounts include 500 mg, 400 mg, 300 mg, 200 mg and 100 mg.

According to a third embodiment of this aspect of the invention, the method of treating iron deficiency comprises the administration of the iron carbohydrate complex, in particular FCM, which is characterized in that the total amount of elemental iron administered within a period of 12 months does not exceed 5000 mg. Preferably, the total amount of elemental iron administered within a period of 12 months does not exceed 4000 mg, 3000 mg or 2000 mg.

### II. Selected Subgroups of Subjects

The methods of the invention are typically performed on a subject in need thereof. A subject in need of the methods of the invention is a subject having, diagnosed with, suspected of having, or at risk for developing iron deficiency, in particular, iron deficiency associated with chronic blood loss, acute blood loss, pregnancy, childbirth, lactation, childhood development, heavy uterine bleeding, menstruation, gastrointestinal bleeding, chronic internal bleeding, inflammatory bowel disease, congestive heart failure, restless leg syndrome, parasitic infections, lost or impaired kidney function such as due to chronic kidney disease or kidney failure, dialysis, surgery, chronic ingestion of agents such as alcohol, salicylates, steroids, non-steroidal anti-inflammatory agents, erythropoiesis stimulating agents (ESAs) or drugs inhibiting iron absorption. Iron deficiency anemia (IDA) develops when iron stores are depleted. Patients who suffer from ID may have IDA; patients with IDA necessarily suffer from ID. Methods to diagnose ID an IDA are well established in the art and commonly used in clinical practice.

Subjects having, diagnosed with, suspected of having, or at risk for developing iron deficiency will be given IV iron in the form of an iron carbohydrate complex, in particular ferric carboxymaltose, if oral iron is not tolerated or not effective in the subject. Another situation where IV iron is indicated is a need to deliver iron rapidly.

A particular group of subjects that are amenable to treatment according to the present invention is characterized as having a reduced risk for FGF23-mediated side effects.

According to a first embodiment of this aspect of the invention, a subject having a reduced risk for FGF23-mediated side effects has a blood or urine parameter selected from the group consisting of:
(1) normal serum phosphate level, in particular >2.5 mg/dL;
(2) normal serum vitamin D level, in particular 1,25-dihydroxyvitamin D being within the following ranges: Males: <16 years: 24-86 pg/mL, ≥16 years: 18-64 pg/mL, Females: <16 years: 24-86 pg/mL, ≥16 years: 18-78 pg/mL;
(3) normal serum ionized calcium level, in particular 1.16-1.32 mmol/L;
(4) normal serum PTH level, in particular 15-65 pg/mL;
(5) normal fractionary urinary phosphate excretion, in particular an FEPi of 10-20% (0.1-0.2 fraction); and
(6) a combination of (1), (2), (3), (4), and (5).

According to a second embodiment of this aspect of the invention, a subject having a reduced risk for FGF23-mediated side effects has a blood parameter selected from the group consisting of:
(1) normal serum Bone Specific Alkaline Phosphatase level, in particular 6.5-22.4 U/L;
(2) normal serum Alkaline Phosphatase level, in particular 31-140 U/L;
(3) normal serum N-terminal Propeptide of Type I Collagen (PINP) level, in particular 15.13-85.50 ng/mL;
(4) normal serum Carboxy-terminal Collagen Crosslinks (CTx) level, in particular 0.03-1.01 ng/mL; and
(5) a combination of (1), (2), (3) and (4).

According to a third embodiment of this aspect of the invention, a subject having a reduced risk for FGF23-mediated side effects is characterized by the absence of one or more and in particular all of the following exclusion criteria:
(1) having undergone bariatric surgery;
(2) obesity;
(3) cardiac conditions with increased risks of arrhythmias;
(4) primary or secondary hyperparathyroidism;
(5) pulmonary disorders such as asthma or chronic obstructive pulmonary disease (COPD)
(6) genetic diseases leading to hypophosphatemia such as X-linked hypophosphatemia, autosomal dominant hypophosphatemic rickets, autosomal recessive hypophosphatemic rickets;
(7) secondary hypophosphatemia or tumor induced hypophosphatemia;
(8) disorders of the bone, such as for example osteoporosis or osteomalacia; and
(9) being scheduled for surgery within 1 day to two months of the iron administration such as preferably being scheduled for surgery within 1 to 3 weeks of iron administration.

According to a fourth embodiment of this aspect of the invention, a subject having a reduced risk for FGF23-mediated side effects is characterized by normal respiratory capacity measured as maximal respiratory pressure and maximal inspiratory pressure, in particular a maximal respiratory pressure of: Males: ≥117-(0.83× age) cm H₂O, Females: ≥95-(0.57× age) cm H₂O; and/or a maximal inspiratory pressure of: Males: ≥62-(0.15× age) cm H₂O, Females: >62-(0.50× age) cm H₂O.

According to a fifth aspect, a subject having a reduced risk for FGF23-mediated side effects has both blood/urine parameters as disclosed herein and is characterized by the absence of exclusion criteria as disclosed herein.

According to a sixth aspect, a subject having a reduced risk for FGF23-mediated side effects has blood/urine parameters as disclosed herein, is characterized by the absence of exclusion criteria as disclosed herein, and is characterized by normal respiratory capacity as disclosed herein.

A further particular group of subjects that are amenable to treatment according to the present invention are subjects with chronic kidney disease (CKD). Frequencies of hypophosphatemia following iron treatment are much lower in patients with chronic kidney disease (CKD), who have impaired renal function and thus impaired ability to excrete phosphate in the urine and as a result tend to rather suffer from hyperphosphatemia (too high phosphate). On the other hand, due to the tendency towards hyperphosphatemia, CKD patients tend to have very high FGF23 levels function due to ongoing attempts of the body to compensate for the high serum phosphate levels by producing iFGF23 to increase the urinary fractional excretion of phosphate via the kidneys. For this reason, iFGF23 levels are elevated in CKD patients, and as a result other downstream effects of iFGF23 may be more pronounced despite the lower risk of hypophosphatemia.

### B. Diagnostic Methods

Further described herein are methods of monitoring a subject who has been administered a first dose of an iron carbohydrate complex, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject is eligible for being administered a second dose of the iron carbohydrate complex if the at least one blood or urine parameter is as disclosed herein.

Further described herein are methods of monitoring a subject who has been administered a first dose of ferric carboxymaltose, comprising determining in a biological sample obtained from the subject at least one blood parameter selected from the group consisting of (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject is eligible for being administered a second dose of ferric carboxymaltose if the at least one blood parameter is normal as disclosed herein.

Still further described herein are methods of monitoring a subject who has been administered a first dose of ferric carboxymaltose, comprising determining the respiratory capacity of the subject, wherein the subject is eligible for being administered a second dose of ferric carboxymaltose if the respiratory capacity is normal as disclosed herein.

Also described herein are methods of identifying a subject suitable for the therapeutic methods of the invention. Such methods include methods of identifying a subject having a reduced risk for FGF23-mediated side effects, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject has a reduced risk for FGF23-mediated side effects if the at least one blood or urine parameter is normal as disclosed herein.

Further described herein are methods of identifying a subject having a reduced risk for FGF23-mediated side effects, comprising determining in a biological sample obtained from the subject at least one blood parameter selected from the group consisting (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject has a reduced risk for FGF23-mediated side effects if the at least one blood or urine parameter is normal as disclosed herein.

Still further described herein are methods of identifying a subject having a reduced risk for FGF23-mediated side effects, comprising determining whether the subject is characterized by one or more and in particular all of the following exclusion criteria:
(1) having undergone bariatric surgery;
(2) obesity;
(3) cardiac conditions with increased risks of arrhythmias;
(4) primary or secondary hyperparathyroidism;
(5) pulmonary disorders such as asthma or chronic obstructive pulmonary disease (COPD)
(6) genetic diseases leading to hypophosphatemia such as X-linked hypophosphatemia, autosomal dominant hypophosphatemic rickets, autosomal recessive hypophosphatemic rickets;
(7) secondary hypophosphatemia or tumor induced hypophosphatemia;
(8) disorders of the bone, such as for example osteoporosis or osteomalacia; and
(9) being scheduled for surgery within 1 day to two months of the iron administration, wherein the subject has a reduced risk for FGF23-mediated side effects if the subject is characterized by the absence of one or more and in particular all of the exclusion criteria.

And yet described herein are methods of identifying a subject having a reduced risk for FGF23-mediated side effects, comprising determining the respiratory capacity of the subject, wherein the subject has a reduced risk for FGF23-mediated side effects if the respiratory capacity is normal as disclosed herein.

The biological sample can be any sample obtained from a subject, wherein the sample allows determining said blood or urine parameters. In some embodiments, the biological sample comprises a blood sample. In some embodiments, the biological sample comprises a plasma sample. In preferred embodiments, the biological sample comprises a serum sample.

In other preferred embodiments, the biological sample comprises a urine sample. As the diagnostic methods are performed on a sample of the subject, the methods are carried out ex vivo, in particular in vitro.

The biological sample can be collected and/or obtained by any method known in the art. In some embodiments, the biological sample is obtained directly from a subject, e.g., by withdrawing the sample directly from the circulatory system of a subject. In other embodiment, the biological sample is obtained from a lab, wherein the lab, or a predecessor, previously obtained the biological sample directly from a subject. In some embodiments, the biological sample is fresh, e.g., the sample has not been frozen or stored for an extended period of time. In other embodiments, the biological sample has been stored at a temperature less than 37° C.

The diagnostic methods of monitoring and identifying a subject according to the invention, in some embodiments, further comprises administering the iron carbohydrate complex, in particular ferric carboxymaltose, to a subject identified as being eligible of being administered a second dose of the iron carbohydrate complex and/or as having a reduced risk for FGF23-mediated side effects. The disclosure herein in relation to the administration regimens and selected subgroups of patients is applicable in this regard.

### C. Drug Combinations

Further described herein are combinations of FCM with one or more additional drugs for use in treating iron deficiency, wherein the additional drug is selected from the group consisting of:
(1) vitamin Ds;
(2) phosphates; and
(3) anti-FGF23 antagonistic antibodies.

FCM leads to a decrease in active vitamin D, 1,25-dihydroxyvitamin D, and to an increase of 24,25-dihydroxyvitamin D. Administering therapeutically effective amounts of vitamin D can help reducing this effect. To this end, administration of alfacalcidol and in particular calcitriol is preferred. Alternatively, cholecalciferol or ergocalciferol may be administered.

If alfacalcidol or calcitriol is administered it may be administered within three days prior to the administration of the first FCM dose. Alternatively, alfacalcidol or calcitriol administration is started on the same day of first FCM dose. Still a further alternative is to start alfacalcidol or calcitriol administration on day 1, day 2, day 3, day 4, day 5, day 6 or day 7, or day 14 after the administration of the first FCM dose.

Calcitriol is expediently administered in a daily dose of 0.125 μg to 2 μg, such as 0.125 μg to 1 μg, such as 0.25 μg-0.75 μg, for example 0.50 μg.

Alfacalcidol is expediently administered in daily doses of 0.25 μg to 5 μg, such as 0.5 μg to 2 μg, such as 1 μg.

Whether administered as pre-treatment to FCM administration or subsequent to FCM administration, treatment with calcitriol or alfacalcidol is continued for until blood parameters, in particular vitamin D level, are normal or until three weeks, four weeks, five weeks, or six weeks after initiation of treatment, whichever occurs earlier.

Cholecalciferol or ergocalciferol is preferably administered prior to treatment with FCM, such as for a period of 14 days, 7 days, 6 days, alternatively 5 days, 4 days, 3 days, 2 days, 1 day prior to administration of the first FCM dose.

Cholecalciferol is expediently administered in weekly doses from 140 μg to 2500 μg, such as preferably 300 μg-600 μg such as preferably 500 μg.

Ergocalciferol is expediently administered in daily doses of 10 μg to 1250 μg, such as preferably 500 μg.

In one embodiment of the invention, the pre-treatment with either cholecalciferol or ergocalciferol prior to administration of the first FCM dose as disclosed herein is followed by treatment with alfacalcidol or calcitriol subsequent to administration of the first FCM dose as disclosed herein.

Because FCM also leads to a decrease in serum phosphate, administering therapeutically effective amounts of phosphates can help reducing this effect. For example, glucose-1-phosphate or a phosphate salt such as calcium phosphate, potassium phosphate or sodium phosphate can be administered orally or intravenously (IV).

Therapeutically effective amounts of phosphate for IV administration include single doses, preferably singly daily doses, of 10 mmol to 50 mmol, such as 30 mmol to 40 mmol and, in particular, 15 mmol to 35 mmol of phosphate, which may be repeated until serum phosphate have normalized.

Therapeutically effective amounts of phosphate for oral administration include single doses, preferably singly daily doses, of 15 mmol to 85 mmol, such as 30 mmol to 65 mmol and, in particular, 45 mmol to 50 mmol of phosphate, which may be repeated until serum phosphate have normalized.

According to a particular embodiment of the invention, a combination of additional drugs is used. The combination of a vitamin D and a phosphate is a particularly preferred combination.

Anti-FGF23 antibodies are used to treat very rare diseases such as x-linked hypophosphatemia. The first such product to reach the market is burosumab. It is preferred that anti-FGF23 antibodies are used in patients experiencing very severe FGF23 mediated side effects following the administration of FCM such as serum phosphate levels below 1 mg/dL or iFGF23 levels which are increased by 100 μg/mL or more relative to baseline.

According to a preferred embodiment, the additional drug is administered orally. This is in particular expedient if the additional drug is a phosphate, such as glucose−1-phosphate calcium phosphate or sodium phosphate, or a vitamin D, such as calcitriol.

According to a particular embodiment, the additional drug is administered prior to the first dose of FCM administration. This is in particular expedient if the additional drug is cholecalciferol or ergocalciferol.

According to another particular embodiment, the additional drug is administered after the first dose but prior to the second dose of FCM administration. This is in particular expedient if the additional drug is calcitriol or alfacalcidol. If phosphates used as additional drugs, it is likewise preferred to administer them after the first dose of FCM administration.

Dosages for the additional drugs usually refer to amounts of drug administered to adults. Dosages for administration to infants may be adjusted accordingly.

### Exemplary Embodiments

1. A method of treating iron deficiency, which comprises administering a first dose and a second dose of ferric carboxymaltose, wherein the time between the first and the second dose is at least 10 days.
2. The method of embodiment 1, wherein the time between the first and the second dose is at least 14 days, 18 days, 21 days, 28 days, 35 days, 42 days, 49 days, or 56 days.
3. The method of embodiment 1, wherein the time between the first and the second dose is at least 3 months or 6 months.
4. The method of any one of embodiments 1-3, wherein the first and the second dose is a single daily dose.
5. The method of any one of embodiments 1-4, wherein the first dose is 750 mg of elemental iron and the second dose is 750 mg of elemental iron.
6. A method of treating iron deficiency, which comprises administering a first dose and a second dose of ferric carboxymaltose, wherein the first and the second dose each do not exceed, or are less than, 500 mg of elemental iron.
7. A method of treating iron deficiency, which comprises administering one or more doses of ferric carboxymaltose, wherein the total amount of elemental iron administered within a period of 12 months does not exceed 5000 mg.
8. A method of treating iron deficiency, which comprises administering ferric carboxymaltose to a subject, wherein the subject has a reduced risk for FGF23-mediated side effects.
9. The method of embodiment 8, wherein the subject having a reduced risk for FGF23-mediated side effects has a blood or urine parameter selected from the group consisting of:
(1) normal serum phosphate level, in particular >2.5 mg/dL;
(2) normal serum vitamin D level, in particular 1,25-dihydroxy vitamin D being within the following ranges: Males: <16 years: 24-86 μg/mL, >16 years: 18-64 μg/mL, Females: <16 years: 24-86 μg/mL, >16 years: 18-78 μg/mL;
(3) normal serum ionized calcium level, in particular 1.16-1.32 mg/dL;
(4) normal serum PTH level, in particular 15-65 μg/mL;
(5) normal fractionary urinary phosphate excretion, in particular an FEPi of 10-20% (0.1-0.2 fraction); and
(6) a combination of (1), (2), (3), (4) and (5).
   10. The method of embodiment 8, wherein the subject having a reduced risk for FGF23-mediated side effects has a blood parameter selected from the group consisting of: (1) normal serum Bone Specific Alkaline Phosphatase level, in particular 6.5-22.4 U/L;
(2) normal serum Alkaline Phosphatase level, in particular 31-40 U/L;
(3) normal serum N-terminal Propeptide of Type I Collagen (PINP) level, in particular 15.13-85.50 ng/mL;
(4) normal serum Carboxy-terminal Collagen Crosslinks (CTx) level, in particular 0.03-1.01 ng/mL; and
(5) a combination of (1), (2), (3) and (4).
   11. The method of embodiment 8, wherein the subject having a reduced risk for FGF23-mediated side effects is characterized by the absence of one or more and in particular all of the following exclusion criteria:
(1) having undergone bariatric surgery;
(2) obesity;
(3) cardiac conditions with increased risks of arrhythmias;
(4) primary or secondary hyperparathyroidism;
(5) pulmonary disorders such as asthma or chronic obstructive pulmonary disease (COPD)
(6) genetic diseases leading to hypophosphatemia such as X-linked hypophosphatemia, autosomal dominant hypophosphatemic rickets, autosomal recessive hypophosphatemic rickets;
(7) secondary hypophosphatemia or tumor induced hypophosphatemia;
(8) disorders of the bone, such as for example osteoporosis or osteomalacia; and
(9) being scheduled for surgery within 1 day to two months of the iron administration.
   12. The method of embodiment 8, wherein the subject having a reduced risk for FGF23-mediated side effects is characterized by normal respiratory capacity measured as maximal respiratory pressure and/or maximal inspiratory pressure, in particular a maximal respiratory pressure of Males: ≥117-(0.83× age) cm H₂O, Females: ≥95-(0.57× age) cm H₂O; and/or a maximal inspiratory pressure of Males: ≥62-(0.15× age) cm H₂O, Females: ≥62-(0.50× age) cm H₂O.
   13. The method of any one of embodiments 1-7 for treating a subject having a reduced risk for FGF23-mediated side effects as defined in embodiments 8-12.
   14. A method of monitoring a subject who has been administered a first dose of ferric carboxymaltose, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject is eligible for being administered a second dose of ferric carboxymaltose if the at least one blood or urine parameter is as defined in embodiment 9.
   15. A method of monitoring a subject who has been administered a first dose of ferric carboxymaltose, comprising determining in a biological sample obtained from the subject at least one blood parameter selected from the group consisting of (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject is eligible for being administered a second dose of ferric carboxymaltose if the at least one blood parameter is as defined in embodiment 10.
   16. A method of monitoring a subject who has been administered a first dose of ferric carboxymaltose, comprising determining the respiratory capacity of the subject, wherein the subject is eligible for being administered a second dose of ferric carboxymaltose if the respiratory capacity is normal.
   17. A method of identifying a subject having a reduced risk for FGF23-mediated side effects, comprising determining in a biological sample obtained from the subject at least one blood or urine parameter selected from the group consisting of (1) serum phosphate level, (2) serum vitamin D level, (3) serum ionized calcium level, (4) serum PTH level and (5) fractionary urinary phosphate excretion, wherein the subject has a reduced risk for FGF23-mediated side effects if the at least one blood or urine parameter is as defined in embodiment 9.
   18. A method of identifying a subject having a reduced risk for FGF23-mediated side effects, comprising determining in a biological sample obtained from the subject at least one blood parameter selected from the group consisting (1) serum Bone Specific Alkaline Phosphatase level; (2) serum Alkaline Phosphatase level, (3) serum N-terminal Propeptide of Type I Collagen (PINP) level and (4) serum Carboxy-terminal Collagen Crosslinks (CTx) level, wherein the subject has a reduced risk for FGF23-mediated side effects if the at least one blood parameter is as defined in embodiment 10.
   19. A method of identifying a subject having a reduced risk for FGF23-mediated side effects, comprising determining whether the subject is characterized by one or more and in particular all of the following exclusion criteria:
(1) having undergone bariatric surgery;
(2) obesity;
(3) cardiac conditions with increased risks of arrhythmias;
(4) primary or secondary hyperparathyroidism;
(5) pulmonary disorders such as asthma or chronic obstructive pulmonary disease (COPD)
(6) genetic diseases leading to hypophosphatemia such as X-linked hypophosphatemia, autosomal dominant hypophosphatemic rickets, autosomal recessive hypophosphatemic rickets;
(7) secondary hypophosphatemia or tumor induced hypophosphatemia;
(8) disorders of the bone, such as for example osteoporosis or osteomalacia; and
(9) being scheduled for surgery within 1 day to two months of the iron administration, wherein the subject has a reduced risk for FGF23-mediated side effects if the subject is characterized by the absence of one or more and in particular all of said exclusion criteria.
   20. A method of identifying a subject having a reduced risk for FGF23-mediated side effects, comprising determining the respiratory capacity of the subject, wherein the subject has a reduced risk for FGF23-mediated side effects if the respiratory capacity is normal.
   21. A combination of ferric carboxymaltose with one or more additional drugs for use in the treatment of iron deficiency, wherein the additional drug is selected from the group consisting of
(1) vitamin Ds;
(2) phosphates; and
(3) anti-FGF23 antagonistic antibodies.
   22. The combination of embodiment 21, wherein the vitamin D is calcitriol, alfacalcidol, cholecalciferol or ergocalciferol.
   23. The combination of embodiment 22, wherein calcitriol or alfacalcidol is administered within three days prior to the administration of the first FCM dose.
   24. The combination of embodiment 22, wherein administration of calcitriol or alfacalcidol is started on the same day of first FCM dose.
   25. The combination of embodiment 22, wherein calcitriol or alfacalcidol administration is started on day 1, day 2, day 3, day 4, day 5, day 6 or day 7, or day 14 after the administration of the first FCM dose.
   26. The combination of embodiment 22, wherein cholecalciferol or ergocalciferol is administered for a period of 14 days, 7 days, 6 days, alternatively 5 days, 4 days, 3 days, 2 days, 1 day prior to administration of the first FCM dose.
   27. The combination of embodiments 22, wherein cholecalciferol or ergocalciferol is administered prior to administration of the first FCM dose followed by treatment with calcitriol or alfacalcidol subsequent to administration of the first FCM dose.
   28. The combination of any one of embodiments 21-27, wherein the additional drug is administered orally.
   29. The combination of any one of embodiment 21-28, wherein the additional drug is administered prior to the second dose of ferric carboxymaltose administration.
   30. The combination of embodiments 21-29, for use in a method of any one of embodiments 1-13.

The present disclosure is further illustrated by the following examples, which should not be construed as further limiting. The contents of all figures and all references, Genbank sequences, patents and published patent applications cited throughout this application are expressly incorporated herein by reference.

### Examples

Two prospective randomized, open-label, comparative trials were performed comparing the incidence of hypophosphatemia in relation to treatment with iron isomaltoside (“IIM”, tradename Monofer®, Monoferric®) and ferric carboxymaltose (“FCM”, trade name Injectafer, Ferinject) in adult human subjects with iron deficiency anaemia.

### Trial Design

The trial was a randomized, open-label, comparative trial. Subjects with Iron Deficiency Anaemia (IDA) were randomized 1:1 to one treatment course of one of the following treatments:
Group A: iron isomaltoside 1000 (Monofer®, Pharmacosmos, Holbok, Denmark, termed iron isomaltoside in the following), 1000 mg at baseline
Group B: ferric carboxymaltose (Ferinject®/Injectafer®, Vifor Inc, Switzerland), 750 mg at baseline and day 7, cumulative dose: 1500 mg

FCM was administered in two single doses of 750 mg of elemental iron one week apart according to its US label. Iron isomaltoside 1000 was administered as a single dose of 1000 mg of elemental iron.

### Objectives

The primary objective of the trial was to compare the incidence of hypophosphatemia in subjects with IDA treated with iron isomaltoside or ferric carboxymaltose.

The secondary safety objective of the trial was to compare the effects of iron isomaltoside and ferric carboxymaltose treatment in subjects with IDA on the following:
(1) Incidence of severe hypophosphatemia
(2) Time with hypophosphatemia
(3) Proportion of subjects with hypophosphatemia at the last visit
(4)S-phosphate (absolute [Δ] and relative [%] changes)
(5) Fractional phosphate urinary excretion
(6) Intact Fibroblast Growth Factor 23 (iFGF23), C-terminal FGF23 (cFGF23), vitamin D (25, 1, 25, 24, 25), Parathyroid Hormone (PTH), and ionized calcium
(7) Adverse Events (AEs) and biochemical safety parameters

The secondary efficacy objective of the trial was to compare the effects of iron isomaltoside and ferric carboxymaltose treatment in subjects with IDA on Haemoglobin (Hb), s-ferritin, and Transferrin Saturation (TSAT).

In addition to the primary and secondary objectives, exploratory analyses on the effect of iron isomaltoside and ferric carboxymaltose were performed including the following
(1) Biochemical bone/muscle markers
(2) Muscle strength

### Endpoints

The primary endpoint was the incidence of hypophosphatemia (defined as s-phosphate <2 mg/dL) at any time from baseline to day 35.

The secondary safety endpoints were the following:
Incidence of s-phosphate below 1.0 mg/dL at any time from baseline to day 35
Time with hypophosphatemia (i.e. time with s-phosphate <2.0 mg/dL) from baseline to day 35
Proportion of subjects with hypophosphatemia at day 35
Absolute [Δ] and relative [%] changes in s-phosphate from baseline to 1, 7, 8, 14, 21, and 35
Fractional phosphate urinary excretion at 1, 7, 8, 14, 21, and 35
Change in iFGF23, cFGF23, vitamin D (25, 1, 25, 24, 25), PTH, and ionized calcium from baseline to 1, 7, 8, 14, 21, and 35
Type and incidence of AEs
Serious or severe hypersensitivity reaction starting on or after the first dose of randomized treatment (i.e. treatment emergent). The hypersensitivity terms were defined as standardised Medical Dictionary for Regulatory Activities query (SMQ) terms.

In addition, physical examinations and measurements of vital signs, height, weight, electrocardiogram (ECG), and safety laboratory parameters were measured as part of standard safety assessments.

The secondary efficacy endpoints were the following:
Change in Hb, s-ferritin, and TSAT from baseline to day 1, 7, 8, 14, 21, and 35 The exploratory endpoints were the following:
Change in biochemical bone/muscle markers (serum N-terminal Propeptide of Type I Collagen (PINP), Carboxy-terminal Collagen Crosslinks (CTx), s-alkaline phosphatase (bone specific and total), and creatine kinase) from baseline to day 1, 7, 8, 14, 21, and
Change in fatigue symptoms from baseline to day 14 and 35 measured by the Functional Assessment of Chronic Illness Therapy (FACIT) Fatigue Scale
Change in QoL from baseline to day 14 and 35 measured by Short Form (SF)-36 questionnaire
Change in bone pain from baseline to day 14 and 35 measured on a Visual Analogue Scale (VAS)
Change in muscle strength from baseline to day 14 and 35 measured by grip strength
Change in upper and lower limb proximal muscle function from baseline to day 14 and 35 measured by the “1 kg arm lift” test and the “30 sec chair stand” test.
Change in respiratory muscles strength from baseline to day 14 and 35 measured by Maximal Inspiratory Pressure (MIP) and Maximal Expiratory Pressure (MEP)

### Safety Assessments

The trial included the following safety assessments:
Measurements of s-phosphate (blood and urine), iFGF23, cFGF23, vitamin D (25, 1, 25, 24, 25), PTH, and ionized calcium
AEs will be collected and evaluated for relatedness, severity, seriousness, and expectedness. They will be reported to authorities and followed-up according to international and local requirements
Physical examinations, measurements of vital signs, ECG, height, weight, and safety laboratory parameters

### Efficacy Assessments

The trial included the following efficacy assessments:
Hb, s-ferritin, TSAT, and s-iron

### Exploratory Assessments

The exploratory assessments included the following:
Measurement of serum N-terminal PINP, CTx, s-alkaline phosphatase (bone specific and total), and creatine kinase
MIP and MEP

### Trial Duration and Number of Visits

For the individual subject, duration of the trial was 5 weeks (including a 28 days screening period) and each subject attended 8 visits.

### Subject Population

Subjects, who fulfilled the following eligibility criteria, were included.

Inclusion Criteria:

A subject will be eligible for inclusion in the trial if he/she fulfils the following criteria:
1. Men or women >18 years having IDA caused by different aetiologies* such as abnormal uterine bleeding, gastrointestinal diseases, cancer, bariatric procedures (gastric bypass operations), and other conditions leading to significant blood loss
2. Hb≤11 g/dL
3. Body weight >50 kg
4. S-ferritin ≤100 ng/mL
5. Estimated Glomerular Filtration Rate (eGFR) ≥65 mL/min/1.73 m²
6. S-phosphate >2.5 mg/dL
7. Documented history of intolerance or unresponsiveness to oral iron therapy** for at least one month*** prior to trial enrolment
8. Willingness to participate and signing the Informed Consent Form (ICF)
The aetiology (also if unknown) for IDA was documented in the medical history and verified in the source documents.
   The intolerance and non-response to oral iron treatment was documented with sign and symptoms in the medical history and verified in the source document.
      There was a documentation of intolerance or unresponsiveness to at least one month of prescribed oral iron therapy per investigator's judgment within the last 9 months and they would not be candidates for oral iron again.

Exclusion Criteria:

A subject was not eligible for inclusion in this trial if he/she fulfilled any of the following criteria:
1. Acute bleeding >500 mL within 72 hours
2. Anaemia predominantly caused by factors other than IDA according to Investigator's judgment
3. Hemochromatosis or other iron storage disorders
4. Known hypersensitivity reaction to any component of iron isomaltoside or ferric carboxymaltose
5. Previous serious hypersensitivity reactions to any IV iron compounds
6. Treatment with IV iron within the last 30 days prior to screening
7. Treatment with erythropoietin or erythropoietin-stimulation agents, red blood cell transfusion, radiotherapy, and/or chemotherapy within the last 30 days prior to screening
8. Received an investigational drug within the last 30 days prior to screening
9. Planned surgical procedure within the trial period
10. Alanine Aminotransferase (ALAT) and/or Aspartate Aminotransferase (ASAT) >3 times upper limit of normal (e.g. decompensated liver cirrhosis or active hepatitis)
11. Surgery under general anaesthesia within the last 30 days prior to screening
12. Any non-viral infection within the last 30 days prior to screening
13. Alcohol or drug abuse within the past 6 months
14. Untreated hyperparathyroidism
15. Kidney transplantation
16. Estimated life expectancy of <6 months or, for cancer patients, an Eastern Cooperative Oncology Group (ECOG) performance status >1
17. Conditions that interfere with the subject's ability to understand the requirements of the trial and/or presumable non-compliance
18. Any other laboratory abnormality, medical condition, or psychiatric disorders which, in the opinion of the Investigator, will put the subject's disease management at risk or may result in the subject being unable to comply with the trial requirements
19. Pregnant or nursing women. In order to avoid pregnancy, women of childbearing potential have to use adequate contraception (e.g. intrauterine devices, hormonal contraceptives, or double barrier method) during the whole trial period and 7 days after the last dosing
   A summary of the subject population included is provided in the following table:

| | IDA04 | | | | IDA05 | | | |
|---|---|---|---|---|---|---|---|---|
| | Iron | | Ferric | | Iron | | Ferric | |
| | Isomaltoside | | Carboxymaltose | | Isomaltoside | | Carboxymaltose | |
| Safety Analysis | 63 | (100.0) | 60 | (100.0) | 62 | (100.0) | 57 | (100.0) |
| Set (N, %) | | | | | | | | |

Age (years)

| | | | | | | | | |
|---|---|---|---|---|---|---|---|---|
| Mean (SD) | 43.9 | (10.4) | 46.3 | (11.6) | 42.2 | (12.9) | 43.1 | (11.5) |

| | | | | |
|---|---|---|---|---|
| Median | 44.0 | 45.5 | 41.0 | 44.0 |
| Min-Max | 25-74 | 27-77 | 19-79 | 20-76 |

Sex (N, %)

| | | | | | | | | |
|---|---|---|---|---|---|---|---|---|
| Female | 61 | (96.8) | 57 | (95.0) | 58 | (93.5) | 54 | (94.7) |
| Male | 2 | (3.2) | 3 | (5.0) | 4 | (6.5) | 3 | (5.3) |

Race (N, %)

| | | | | | | | | |
|---|---|---|---|---|---|---|---|---|
| White | 38 | (60.3) | 38 | (63.3) | 28 | (45.2) | 29 | (50.9) |
| Asian | 2 | (3.2) | 1 | (1.7) | | | | |
| Black or African | 22 | (34.9) | 19 | (31.7) | 32 | (51.6) | 27 | (47.4) |
| American | | | | | | | | |
| Other | 1 | (1.6) | 2 | (3.3) | 1 | (3.2) | 1 | (1.8) |

### Trial Treatment

The subjects were dosed with either one treatment course of iron isomaltoside (group A) or one treatment course of ferric carboxymaltose (group B) as described below.
Group A: iron isomaltoside was administered as a single IV infusion of 1000 mg at baseline diluted in 100 mL 0.9% sodium chloride and given over approximately 20 minutes (50 mg iron/min, cumulative dose: 1000 mg).
Group B: ferric carboxymaltose was administered as 750 mg infused over at least 15 minutes at baseline and day 7 (cumulative dose: 1500 mg).

No premedication (e.g. antihistamine or steroids) was allowed before administration of the trial drug. If the subject was in daily treatment for e.g. allergy or asthma this was not considered as “premedication” and could be continued.

### Statistical Analyses

The primary endpoint, incidence of hypophosphatemia (defined as s-phosphate <2 mg/dL) at any time from baseline to day 35, was tabulated and exact 95% CI were estimated for each treatment group.

Iron isomaltoside was compared to ferric carboxymaltose by estimation of the risk difference and the associated 95% CI, adjusting for strata (type of underlying disease (women with IDA due to gynaecological blood losses; yes/no) and screening s-phosphate level (< or >3.5 mg/dL)) using the Cochran-Mantel-Haenszel method.

As to sensitivity, the treatment groups were compared between the treatment groups by a logistic regression model with treatment and type of underlying disease as factors and baseline s-phosphate as covariate and by Fisher's exact tests.

All subjects in the safety analysis set were included in the analysis. The first post-baseline phosphate measurement was taken at day 1; hence, very few missing values are expected. If there were subject(s), for whom no post-baseline phosphate measurement(s) were available, these subjects will be set as having s-phosphate <2 mg/dL in the primary analysis.

All the statistical analyses will be described in a statistical analysis plan.

### Baseline Assessment

| IDA04 | | IDA05 | |
|---|---|---|---|
| Iron | Ferric | Iron | Ferric |
| Isomaltoside | Carboxymaltose | Isomaltoside | Carboxymaltose |
| N (%) | N (%) | N (%) | N (%) |

Disease stratum: IDA due to gynaecological blood losses

| | | | | |
|---|---|---|---|---|
| Yes | 41 (65.1) | 42 (70.0) | 44 (71.0) | 39 (68.4) |
| No | 22 (34.9) | 18 (30.0) | 18 (29.0) | 18 (31.6) |

Disease stratum: Screening s-phosphate level

| | | | | |
|---|---|---|---|---|
| <3.5 mg/dL | 32 (50.8) | 33 (55.0) | 35 (56.5) | 32 (56.1) |
| ≥3.5 mg/dL | 31 (49.2) | 27 (45.0) | 27 (43.5) | 25 (43.9) |

### Trial Assessments

### Demographic and Baseline Assessments

Date of birth, gender, race, ethnicity, and smoking habits were collected. A current smoker was defined as a subject who had been smoking within the last 6 months.

### Pregnancy Test

A urine pregnancy test was performed for all women of childbearing potential. The test was handled and interpreted by the site personnel.

### Relevant Medical History

Relevant medical history was recorded. Changes in medical history were recorded at the subsequent visits during the trial (worsening of symptoms or diseases were recorded as AEs). The following was collected: disease and start and stop date. Except for underlying disorder causing IDA, start dates occurring >12 months before the enrolment into the trial were set as >12 months.

### Concomitant Medication

If the subject was receiving any concomitant medication it was recorded at the baseline visit. Changes in concomitant medication were recorded in the subsequent visits during the trial. The following was collected: brand name, indication, route, dose, frequency, unit, and start and stop date. Start dates occurring >12 months before the enrolment into the trial were set as >12 months.

### Physical Examination

A physical examination was performed based upon the Investigator's judgement and could include the following:
Head-Eyes-Ear-Nose-Throat
Cardiovascular system
Respiratory system
Nervous system
Gastrointestinal system
Musculo-skeletal system
Urogenital system
Dermatology system
Others, if required

### Height

Height was measured without shoes.

### Weight

Weight was measured.

### Vital Signs

Heart rate and blood pressure were measured at the following time points when a subject received trial drug: approximately 0-10 minutes before infusion, during infusion, 5-15 minutes, and 20-40 minutes after the infusion has ended. If vital signs were measured more than once in the given time interval, the lowest measurement of diastolic blood pressure (including the attendant systolic blood pressure and heart rate) for the period was noted in the electronic Case Report Form (eCRF).

### Electrocardiogram

A standard 12 lead ECG was recorded (including date, time, and signature). At baseline and other treatment visits, two ECGs were recorded; one before administration of the trial drug and one approximately 30 minutes after start of the dosing. Only one ECG was recorded at the follow-up visits.
The ECGs did not need to be evaluated by a cardiologist.

### Respiratory Muscles Strength

The measurements of MIP and MEP provided a non-invasive clinical method for evaluating the strength of respiratory muscles, and it was the most widely used test to assess muscle pressures [ATS/ERS statement, 2002]. The MIP reflected the strength of the diaphragm and other inspiratory muscles, while the MEP reflects the strength of the abdominal muscles and other expiratory muscles. MIP and MEP were measured by MicroRPM (CareFusion Germany 234 GmbH, Hoechberg, Germany). Three tests were performed for both MIP and MEP with the highest value from the three tests taken as the achieved result.

### Laboratory Assessments

It was requested that the blood samples were drawn before administering the trial drug, and, if possible, that they were drawn at the same time of the day at all visits in order to reduce any diurnal fluctuation in the parameters.
Laboratory assessments were performed at a central laboratory. A Laboratory Manual was provided to each site in which all laboratory procedures were described.

### Eligibility Laboratory Assessments

The following eligibility laboratory assessments were performed:
Complete haematology set: Hb, leucocytes/White Blood Cells (WBC), erythrocytes/Red Blood Cells (RBC), haematocrit, platelets, neutrophil granulocytes, lymphocytes, monocytes, eosinophils, basophils, Mean Corpuscular Haemoglobin (MCH), Mean Corpuscular Volume (MCV), Mean Corpuscular Haemoglobin Concentration (MCHC), and reticulocyte count
Biochemistry:
   S-ferritin
   S-phosphate
   Alanine Aminotransferase (ALAT) and Aspartate Aminotransferase (ASAT)
   C-reactive Protein (CRP)
   Estimated Glomerular Filtration Rate (eGFR)
   PTH

### Vitamin E

Vitamin E was measured at baseline visit as part of the demographic data.

### Safety Laboratory Assessments

The following safety laboratory assessments were analysed:
S-phosphate: inorganic phosphorous forms an ammonium phosphomolybdate complex having the formula (NH₄)₃[PO4](MoO3)12 with ammonium molybdate in the presence of sulfuric acid. The complex was determined photometrically in the ultraviolet region (340 nm) of the spectrum using the Roche Modular and Cobas Analyzer.
iFGF23 and cFGF23: the human intact FGF23 was measured by the 2nd generation Elisa kit manufactured by Immonotropics, Inc, San Clemente, CA. This was a two-site enzyme-linked immunosorbent assay. The human C-terminal FGF23 was measured by the Elisa kit manufactured by Immonotropics, Inc, San Clemente, CA. This was a two-site enzyme-linked immunosorbent assay.
Vitamin D (25, 1, 25, 24, 25): Following protein precipitation, 25-hydroxyvitamin D2, 25-hydroxyvitamin D3 and their internal standards were extracted by supported liquid extraction (SLE). After evaporation under nitrogen, the residue was reconstituted and analyzed using Liquid Chromatography (LC) with Tandem Mass Spectrometric detection (MS/MS). The standard curve range was 0.5 ng/mL to 200.00 ng/mL using a serum volume of 0.1 mL.
PTH: The Intact PTH assay was performed using the iPTH reagent packs for the ADVIA Centaur XP instruments. The assay was a two-site sandwich immunoassay using direct chemiluminometric technology, which used constant amounts of two anti-human PTH antibodies in the Lite Reagent. The first antibody was a polyclonal goat anti-human PTH (N-terminal 1-34) antibody labeled with acridinium ester. The second antibody was a biotinylated polyclonal goat anti-human PTH (39-84 region) antibody. Streptavidin in the Solid Phase was covalently coupled to paramagnetic latex particles. A direct relationship exists between the amount of PTH present in the patient sample and the amount of relative light units (RLUs) detected by the system.
Ionized calcium: Measured by the IL GEM Premier 3500 PAK cartridge. The central component was the sensor card, which provided a low volume, gas tight chamber in which the sample was presented to the sensors. The pH and electrolyte sensors were all based on the principle of ion-selective electrodes; that is, an electrical potential could be established across a membrane which was selectively permeable to a specific ion. The pH and electrolyte sensors were polyvinyl chloride (PVC) based ion selective electrodes, consisting of an internal Ag/AgCl reference electrode and internal salt layer. The potentials were measured against the card reference electrode.
Complete haematology set: Leucocytes/WBC, erythrocytes/RBC, haematocrit, platelets, neutrophil granulocytes, lymphocytes, monocytes, eosinophils, basophils, MCH, MCV, MCHC, and reticulocyte count
Biochemistry:
   S-sodium, s-potassium, s-calcium, s-urea, s-creatinine, s-albumin
   S-bilirubin, ASAT, ALAT
   CRP

### Efficacy Laboratory Assessments

The following efficacy laboratory parameters were analysed:
Hb
S-ferritin: The Access ferritin assay was a two-site immunoenzymatic (“sandwich” assay). A sample was added to a reaction vessel with goat anti-ferritin-alkaline phosphatase conjugate, and paramagnetic particles coated with goat anti-mouse: mouse anti-ferritin complexes. Serum or plasma (heparin) ferritin binds to the immobilized monoclonal anti-ferritin on the solid phase, while the goat anti-ferritin enzyme conjugate reacts with different antigenic sites on the ferritin molecules. Separation in a magnetic field and washing removed materials not bound to the solid phase. A chemiluminescent substrate, Lumi-Phos* 530, was added to the reaction vessel and light generated by the reaction was measured with a luminometer.
TSAT (s-iron and transferrin will be collected to calculate the TSAT)

### Exploratory Laboratory Assessments

The following exploratory laboratory assessments were analysed:
Serum N-terminal PINP: The measurement method was a sandwich principle, electrochemiluminescence immunoassay (ECLIA). During the first incubation, PINP in the sample and a biotinylated monoclonal PINP-specific antibody were incubated together. During the second incubation, streptavidin-coated labelled microparticles and a monoclonal PINP-specific antibody labelled with a ruthenium complex (Trs (2,2-bipyridyl)ruthenium (II)-complex (Ru(bpy)23+)) were added to form a sandwich complex, which bound to the solid phase via interaction of biotin and streptavidin. The reaction mixture was aspirated into the measuring cell where the microparticles were magnetically captured onto the surface of the electrode. A voltage was applied to the electrode, which induced chemiluminescent emission, which was measured by a photomultiplier. Results were determined via a calibration curve, which was instrument-specifically generated by a 2-point calibration and a master curve provided via the reagent barcode. The method was run on a Cobas e601 Analyzer.
CTx: During the first incubation, 50 μL of sample and biotinylated monoclonal anti-beta-CrossLaps antibody were incubated together. During the second incubation, streptavidin-coated labelled microparticles and a monoclonal P beta-CrossLaps-specific antibody labelled with ruthenium complex were added to form a sandwich complex, which bound to the solid phase via interaction of biotin and streptavidin. The reaction mixture was aspirated into the measuring cell where the microparticles were magnetically captured onto the surface of the electrode. Unbound substances were then removed with ProCell. A voltage was applied to the electrode, which induced chemiluminescent emission, which was measured by a photomultiplier. Results were determined via a calibration curve, which was instrument-specifically generated by a 2-point calibration and a master curve provided via the reagent barcode. The method was run on a Cobas e601 Analyzer.
S-alkaline phosphatase (bone specific and total): Bone Specific Phosphatase (BAP) was measured on Beckman Dxi 800. The Beckman-Coulter Ostase assay was a one-step immunoenzymatic chemiluminescence assay. A mouse monoclonal antibody specific to Bone Specific Alkaline phosphatase (BAP) was added to a reaction vessels with paramagnetic particles coated with goat anti-mouse polyclonal antibody. Calibrators, controls, and samples containing BAP were added to the coated particles and bound to the anti-BAP monoclonal antibody. Chemiluminescent substrate, Lumi-Phos*530, was added to the reaction vessel and light generated by the reaction was measured with a luminometer. The light production is directly proportional to the amount of BAP in the sample. The amount of BAP in the sample was determined by means of a stored multi-point calibration curve. The level of total Alkaline Phosphatase in blood serum was measured using a Roche Modular Analyzer. In the presence of magnesium and zinc ions, p-nitrophenyl phosphate was cleaved by phosphatase into phosphate and p-nitrophenol. The p-nitrophenol released is proportional to the ALP activity and was measured photometrically.
Creatine kinase: The creatine kinase (CK) assay was performed on the Roche Modular and Cobas Analyzers. The reaction proceeded as follows:
   Creatine phosphate+ADP CK >creatine+ATP
   ATP+glucose HK >ADP+G-6-P
   G-6-P+NADP+>G-6-PDH 6-PG+NADPH+H+
The formation of NADPH proceeded at the same rate as the formation of creatine in equimolar amounts. The rate of NADPI formation is proportional to CK activity and is measured photometrically

### Urine Assessments

A spot urine sampling was collected in order to assess the level of fractional s-phosphate excretion.

### Adverse Events

AEs were collected and evaluated for relatedness to trial drug, seriousness, severity, and expectedness.

### Results

Key findings include a clear impact of the FCM dosing regimen on the endocrine system managing phosphate, vitamin-D and calcium.

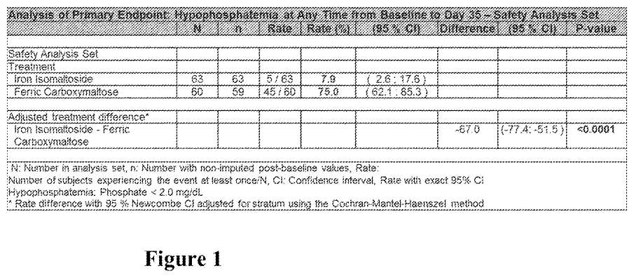

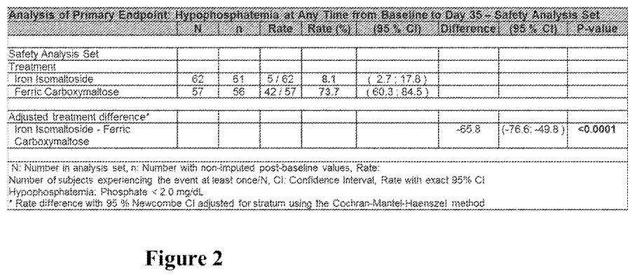

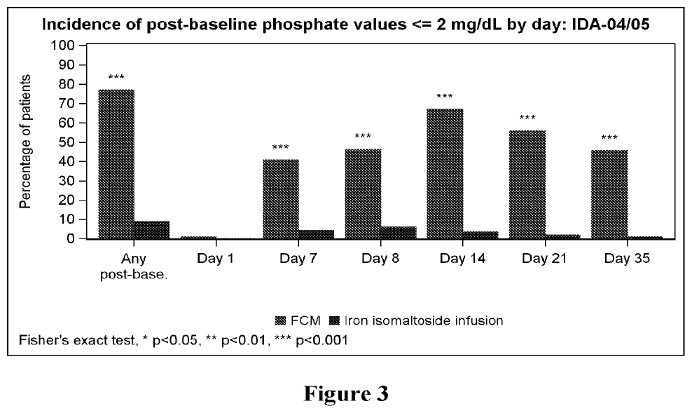

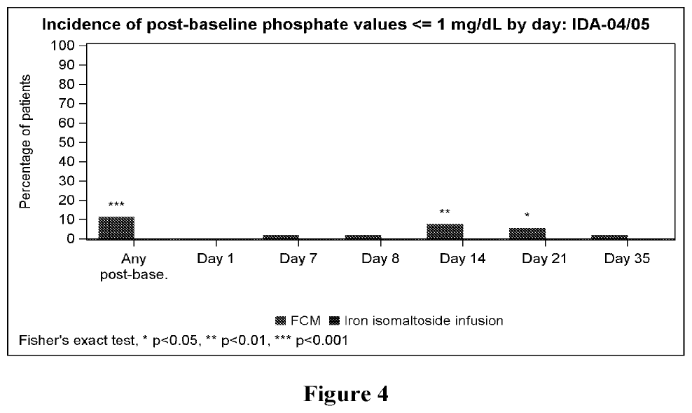

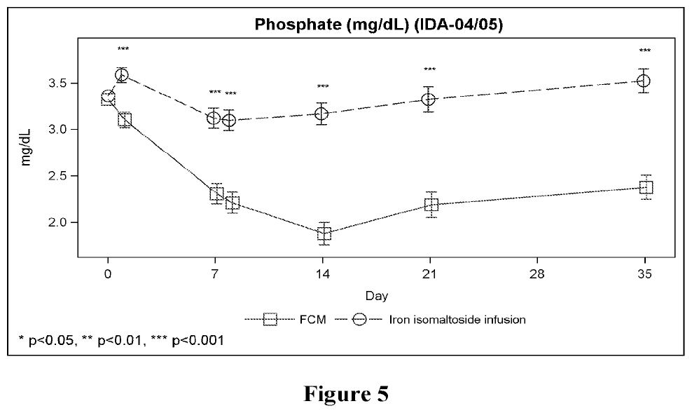

The rate of hypophosphatemia was significantly higher for FCM vs JIM, both in terms of the moderate to severe form with serum phosphate below 2 mg/dL and the severe form of serum phosphate at or below 1.0 mg/dL. See FIGS. 1 to 5.

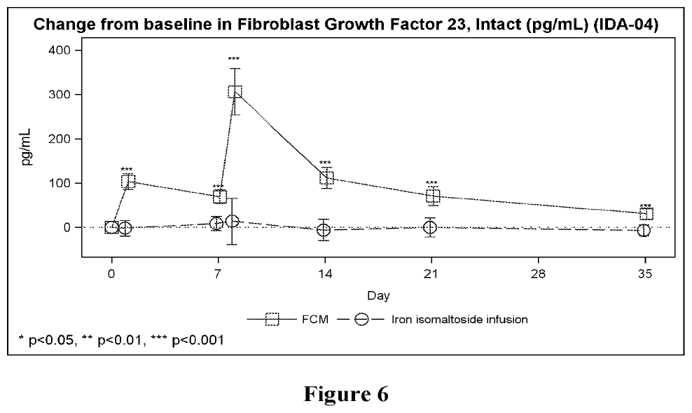

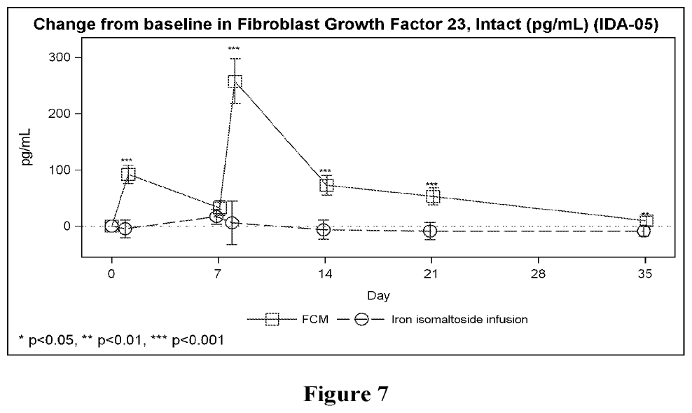

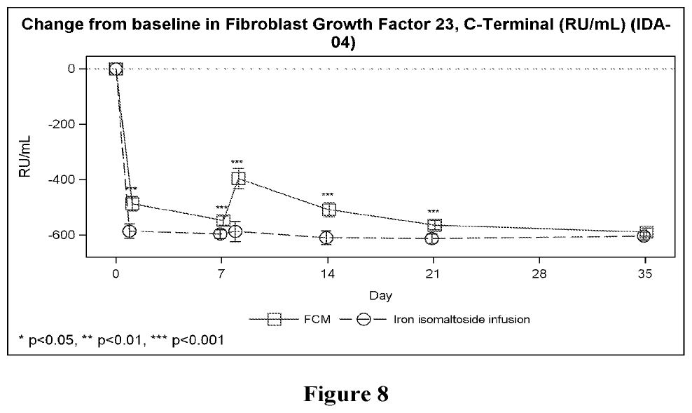

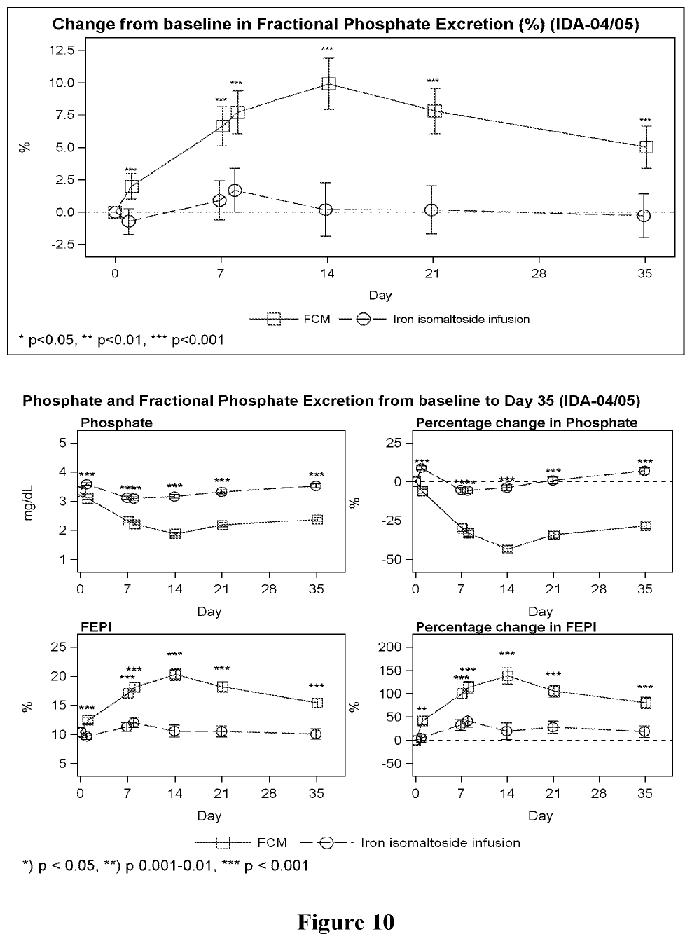

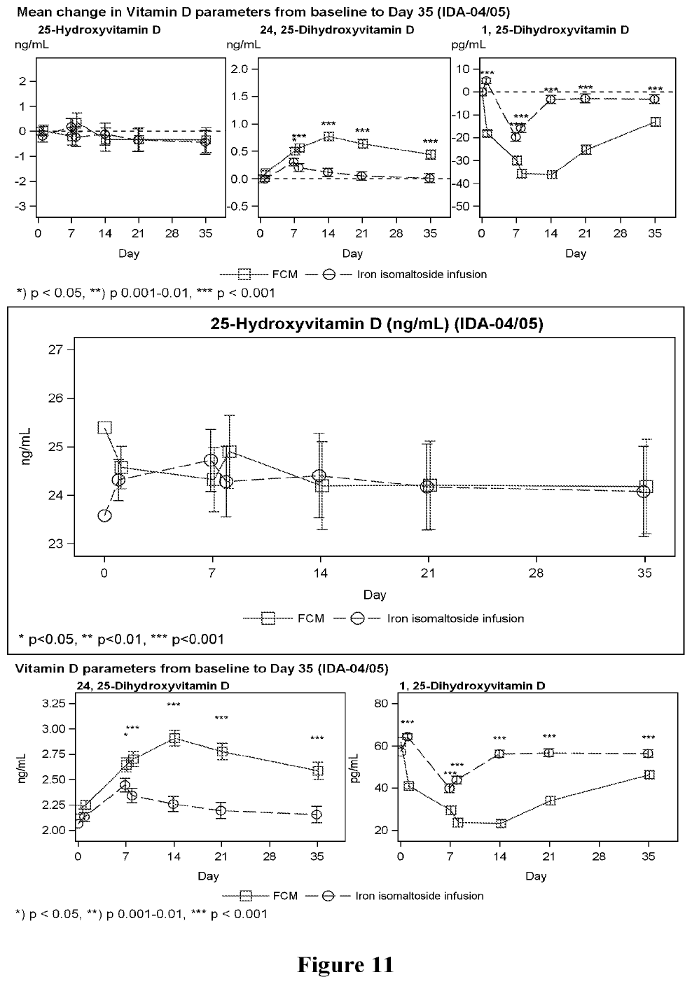

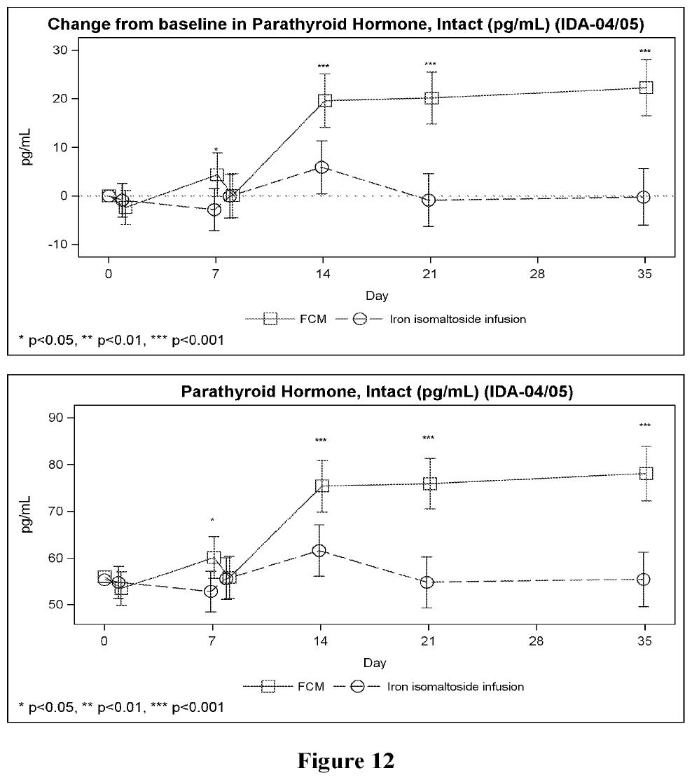

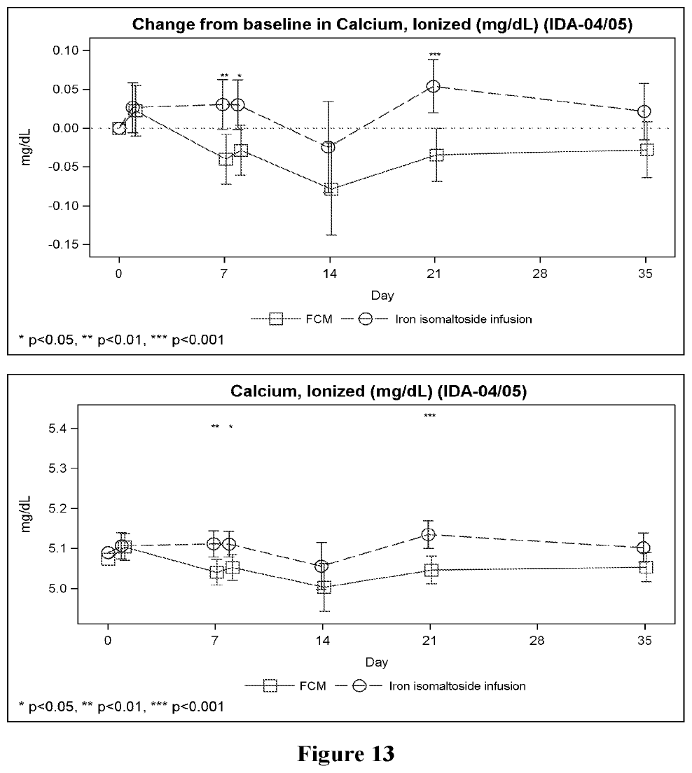

Intact iFGF23 (iFGF23) increased sharply after administration of FCM and then gradually declined over a matter of days. On the second administration, iFGF23 again increased, but this time to a level several fold higher than the initial level, i.e. a surprising and previously unknown self-amplifying effect. For JIM essentially no change of the iFGF23 level was observed. See FIGS. 6 and 7. C-terminal FGF23 (cFGF23) which was initially high due to the underlying iron deficiency anemia dropped sharply on the first administration of FCM whereas it increased on the second administration of FCM. See FIG. 8. Fractional urinary excretion of phosphate was significantly increased for FCM vs IIM. See FIG. 10. FCM led to an increase in PTH, a decrease in 1,25-dihydroxyvitamin D, an increase in 24,25-dihydroxyvitamin D and a decrease in ionized calcium. See FIGS. 11 to 13.

Moreover, FCM led to the significant changes in bone turnover and muscle function as measured by the following biochemical bone/muscle markers.

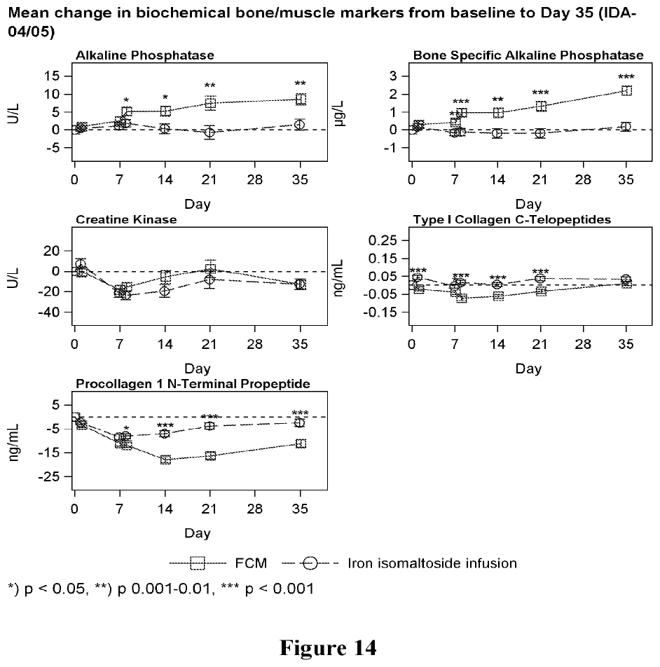

FCM induced a statistically significant increase of Bone Specific Alkaline Phosphatase compared to IMM. See FIG. 14.

FCM induced statistically significant lower N-terminal PINP values than IMM. See FIG. 14.

FCM induced a statistically significant lower CTx values than IMM. See FIG. 14.

FCM induced a statistically significant increase of Alkaline Phosphatase compared to IMM. See FIG. 14.

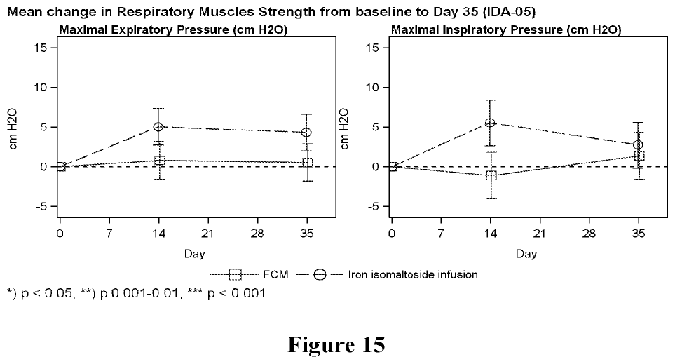

FCM also lead to reduced muscle function relative to the comparator IV iron treatment as specifically measured through respiratory capacity measured as maximal respiratory pressure and/or maximal inspiratory pressure. See FIG. 15.

### EQUIVALENTS

Those skilled in the art will recognize, or be able to ascertain using no more than routine experimentation, many equivalents of the specific embodiments disclosed herein. Such equivalents are intended to be encompassed by the following claims.

### NON-PATENT PUBLICATIONS

Aksan et al., Aliment Pharmacol Ther 2017, 45(10), 1303-1318
Bager et al., Br J Clin Pharmacol 2017, 83, 1118-1125
Bregman et al., Ther Adv Hematol 2014, 5(2), 48-60
Charytan et al., Nephrol Dial Transplant 2013, 28, 953-964
Evstatiev, Gastroenterology 2011, 141, 846-853
Hussain et al., Anemia 2013, Article ID 169107, 10 pages
Ikuta et al., Int J Hematol 2018, https://doi.org/10.1007/s12185-018-2501-8
Klein et al. BMJ Case Rep 2018; doi:10.1136/bcr-2017-222851
Prats et al., BMC Nephrology 2013, 14:167
Qunibi et al., Nephrol Dial Transplant 2011, 26, 1599-1607
Sari et al., Neth J Med 2017, 75(2), 65-73
Schaefer et al., Gastroenterology 2017, 152(6), e5-e6
Seid et al., Am J Obstet Gynecol 2008, 199:435.el-435.e7
Stein et al., Scand J Gastroenterol 2018, https://doi.org/10.1080/00365521.2018.1498914
Van Wyck et al., Transfusion 2009, 49(12), 2719-2728
Wolf et al., Journal of Bone and Mineral Research 2013, 28(8), 1793-1803
Zoller et al., Curr Opin Nephrol Hypertens 2017, 26(4), 266-275

1-15. (canceled)

16. A method of reducing severity of side effects associated with hypophosphatemia in connection with repeated treatment of iron deficiency with ferric carboxymaltose to a subject in need thereof, said method comprising,
administering a dose of ferric carboxymaltose to said subject;
determining that said subject needs a further dose of ferric carboxymaltose;
prior to administering the further dose of ferric carboxymaltose, measuring said subject's serum phosphate levels, and if said subject has hypophosphatemia, treating the hypophosphatemia as medically indicated prior to administering the further dose of ferric carboxymaltose; and
administering the further dose of ferric carboxymaltose to said subject.

17. The method of claim 16, wherein said subject has symptomatic hypophosphatemia.

18. The method of claim 16, wherein said side effects comprise reduced muscle function.

19. The method of claim 16, wherein said side effects comprise increased bone turnover.

20. The method of claim 16, wherein said side effects comprise osteomalacia.

21. The method of claim 16, wherein said side effects comprise fractures of the bone.

22. The method of claim 16, wherein said side effects comprise bone pain.

23. The method of claim 16, wherein said side effects comprise fatigue.

24. The method of claim 16, wherein said subject has inflammatory bowel disease.

25. The method of claim 16, wherein said subject has vitamin D deficiency.

26. The method of claim 16, wherein said subject has iron deficiency anemia.

27. The method of claim 26, wherein said subject has intolerance to oral iron or has had unsatisfactory response to oral iron.

28. The method of claim 26, wherein said subject has lost or impaired kidney function.

29. The method of claim 16, wherein said subject has heart failure.

30. The method of claim 16, wherein the subject has abnormal uterine bleeding.

31. The method of claim 16, wherein measuring said subject's serum phosphate levels comprises measuring the serum phosphate levels in a biological sample obtained from said subject, wherein said subject has hypophosphatemia if their serum phosphate is:
i. <2.5 mg/dL;
ii. <2 mg/dL; or
iii. <1 mg/dL.

32. The method of claim 16, wherein the medically indicated treatment comprises a phosphate selected from glucose−1-phosphate, calcium phosphate, potassium phosphate, or sodium phosphate.

33. The method of claim 32, wherein the phosphate is administered to said subject orally or intravenously (IV).

34. The method of claim 16, wherein the medically indicated treatment comprises a vitamin D selected from calcitriol, alfacalcidol, cholecalciferol or ergocalciferol.

35. The method of claim 16, wherein the side effects are side effects associated with the further dose of ferric carboxymaltose.

36. The method of claim 16, wherein the time between the dose and the further dose is not more than 3 months.

37. The method of claim 16, wherein the further dose of ferric carboxymaltose is part of a repeat course of treatment following a first course of treatment.

38. The method of claim 37, wherein the side effects are side effects associated with the repeat course of treatment.

39. The method of claim 37, wherein the first course of treatment administered to said subject is administered as a single-dose treatment.

40. The method of claim 37, wherein the repeat course of treatment administered to said subject is administered as a single-dose treatment.

41. The method of claim 37, wherein the first course of treatment with ferric carboxymaltose comprises:
a. a dose of ferric carboxymaltose containing 750 mg of elemental iron and a further dose of ferric carboxymaltose containing 750 mg of elemental iron; or
b. a dose of ferric carboxymaltose containing 1000 mg of elemental iron and a further dose of ferric carboxymaltose containing 500 mg to 1000 mg of elemental iron.

42. The method of claim 37, wherein the repeat course of treatment with ferric carboxymaltose contains 500 mg to 1000 mg elemental iron.

43. The method of claim 37, further comprising delaying administration of the repeat course of treatment to a subject exhibiting low serum phosphate levels following the first course of treatment, in order to allow said subject's phosphate levels to normalize prior to the repeat course of treatment.

44. The method of claim 37, wherein the time between the first course of treatment and the repeat course of treatment is not more than 3 months.

45. The method of claim 37, wherein the first course of treatment and the repeat course of treatment are received by the subject within 3 months.

46. A method of reducing severity of side effects associated with hypophosphatemia in connection with repeated treatment of iron deficiency with ferric carboxymaltose to a subject in need thereof, said method comprising,
administering a dose of ferric carboxymaltose to said subject;
determining that said subject needs a further dose of ferric carboxymaltose;
prior to administering the further dose of ferric carboxymaltose, first treating said subject with a medically indicated treatment for hypophosphatemia, then measuring said subject's serum phosphate levels; and
administering the further dose of ferric carboxymaltose to said subject.

47. The method of claim 46, wherein said subject has symptomatic hypophosphatemia.

48. The method of claim 46, wherein said side effects comprise reduced muscle function.

49. The method of claim 46, wherein said side effects comprise increased bone turnover.

50. The method of claim 46, wherein said side effects comprise osteomalacia.

51. The method of claim 46, wherein said side effects comprise fractures of the bone.

52. The method of claim 46, wherein said side effects comprise bone pain.

53. The method of claim 46, wherein said side effects comprise fatigue.

54. The method of claim 46, wherein said subject has inflammatory bowel disease.

55. The method of claim 46, wherein said subject has vitamin D deficiency.

56. The method of claim 46, wherein said subject has iron deficiency anemia.

57. The method of claim 56, wherein said subject has intolerance to oral iron or has had unsatisfactory response to oral iron.

58. The method of claim 56, wherein said subject has lost or impaired kidney function.

59. The method of claim 46, wherein said subject has heart failure.

60. The method of claim 46, wherein the subject has abnormal uterine bleeding.

61. The method of claim 46, wherein measuring said subject's serum phosphate levels comprises measuring the serum phosphate levels in a biological sample obtained from said subject, wherein said subject is only administered the further dose of ferric carboxymaltose if their serum phosphate is:
i. >2.5 mg/dL; or
ii. >2 mg/dL.

62. The method of claim 46, wherein the medically indicated treatment comprises a phosphate selected from glucose−1-phosphate, calcium phosphate, potassium phosphate, or sodium phosphate.

63. The method of claim 62, wherein the phosphate is administered to said subject orally or intravenously (IV).

64. The method of claim 46, wherein the medically indicated treatment comprises a vitamin D selected from calcitriol, alfacalcidol, cholecalciferol or ergocalciferol.

65. The method of claim 46, wherein the side effects are side effects associated with the further dose of ferric carboxymaltose.

66. The method of claim 46, wherein the time between the dose and the further dose is not more than 3 months.

67. The method of claim 46, wherein the further dose of ferric carboxymaltose is part of a repeat course of treatment following a first course of treatment.

68. The method of claim 67, wherein the side effects are side effects associated with the repeat course of treatment.

69. The method of claim 67, wherein the first course of treatment administered to said subject is administered as a single-dose treatment.

70. The method of claim 67, wherein the repeat course of treatment administered to said subject is administered as a single-dose treatment.

71. The method of claim 67, wherein the first course of treatment with ferric carboxymaltose comprises:
a. a dose of ferric carboxymaltose containing 750 mg of elemental iron and a further dose of ferric carboxymaltose containing 750 mg of elemental iron; or
b. a dose of ferric carboxymaltose containing 1000 mg of elemental iron and a further dose of ferric carboxymaltose containing 500 mg to 1000 mg of elemental iron.

72. The method of claim 67, wherein the repeat course of treatment with ferric carboxymaltose contains 500 mg to 1000 mg elemental iron.

73. The method of claim 67, further comprising delaying administration of the repeat course of treatment to a subject exhibiting low serum phosphate levels following the first course of treatment, in order to allow said subject's phosphate levels to normalize prior to the repeat course of treatment.

74. The method of claim 67, wherein the time between the first course of treatment and the repeat course of treatment is not more than 3 months.

75. The method of claim 67, wherein the first course of treatment and the repeat course of treatment are received by the subject within 3 months.
